(19) United States
(12) Patent Application Publication
(10) Pub. No.: US 2023/0212644 A1
(43) Pub. Date: Jul. 6, 2023

(54) IMAGING AND SEQUENCING PROTEIN-DNA INTERACTIONS IN SINGLE CELLS USING INTEGRATED MICROFLUIDICS

(71) Applicants: CHAN ZUCKERBERG BIOHUB, INC., San Francisco, CA (US); THE REGENTS OF THE UNIVERSITY OF CALIFORNIA, Oakland, CA (US)

(72) Inventors: Aaron Streets, Oakland, CA (US); Nicolas Altemose, Oakland, CA (US); Annie Maslan, Oakland, CA (US)

(21) Appl. No.: 17/798,506

(22) PCT Filed: Feb. 9, 2021
(86) PCT No.: PCT/US2021/017260

§ 371 Date: Aug. 9, 2022

Related U.S. Application Data
(60) Provisional application No. 62/972,178, filed on Feb. 10, 2020.

(51) Int. Cl. C12Q 1/6806; B01L 3/00

(52) U.S. Cl. CPC C12Q 1/6806; B01L 3/502707; B01L 2200/10; B01L 2200/0647

(57) ABSTRACT

The present disclosure provides materials and methods for co-determining the cellular location and nucleotide sequence of a DNA that is contacted by (or in close proximity to) a protein of interest in a single cell. Thus the present disclosure provides methods and materials wherein the cellular location of the DNA comprising a DNA-binding site or otherwise in close proximity to a protein of interest is coupled to the sequence of said DNA to provide contemporaneous imaging and sequence measurement of a protein-DNA interaction.

### STATEMENT REGARDING FEDERALLY SPONSORED RESEARCH

The invention was made with Government support under Grant No. GM124916 awarded by NIH National Institute of General Medical Sciences. The government has certain rights in the invention.

### INCORPORATION BY REFERENCE OF MATERIAL SUBMITTED ELECTRONICALLY

The Sequence Listing, which is a part of the present disclosure, is submitted concurrently with the specification as a text file. The name of the text file containing the Sequence Listing is “54663_Seqlisting.txt”, which was created on Feb. 9, 2021 and is 1,951 bytes in size. The subject matter of the Sequence Listing is incorporated herein in its entirety by reference.

### FIELD

The present disclosure relates generally to methods for combining imaging and sequencing information related to protein-DNA interactions in single cells.

### BACKGROUND

Complex life depends on the protein-DNA interactions that constitute and maintain the epigenome, including interactions with histone proteins, transcription factors, DNA (de)methylases, and chromatin remodeling complexes, among others. These interactions enable the static DNA sequence inside the nucleus to dynamically execute different gene expression programs that shape the cell’s identity and behavior. Methods for measuring protein-DNA interactions have proven indispensable for understanding the epigenome, though to date most of this knowledge has been derived from experiments in bulk cell populations. By requiring large numbers of cells, these bulk methods can fail to capture critical epigenomic processes that occur in small numbers of dividing cells, including processes that influence embryo development, developmental diseases, stem cell differentiation, and certain cancers. By averaging together populations of cells, bulk methods also fail to capture important epigenomic dynamics occurring in asynchronous single cells during differentiation or the cell cycle. Because of this, bulk methods can overlook important biological heterogeneity within a tissue. It also remains difficult to pair bulk biochemical data with imaging data, which inherently provide information in single cells, and which can reveal the spatial location of protein-DNA interactions within the nuclei of living cells. These limitations underline the need for high-sensitivity single-cell methods for measuring protein-DNA interactions.

### SUMMARY OF THE INVENTION

One aspect of the present disclosure provides a method for co-determining, e.g., linking, the cellular location and nucleotide sequence of a DNA that is contacted by a protein of interest in a single cell, said method comprising the steps of: (a) incubating a collection of cells that express at least one protein of interest under conditions that allow the at least one protein of interest to contact a DNA sequence; (b) isolating a single cell from the collection of cells and determining the cellular location of the DNA sequence within the single cell; (c) amplifying and collecting the DNA comprising the DNA sequence; and (d) determining the sequence of the DNA sequence; wherein steps (b)-(c) are carried out in separate chambers within one lane of a microfluidic device.

In another aspect, the aforementioned is provided wherein the incubating step (a) is carried out in a chamber within one lane of the microfluidic device.

In still another aspect, an aforementioned method is provided wherein the DNA sequence comprises a DNA-binding site. In still another aspect, an aforementioned method is provided wherein the cells have been induced to express the protein of interest. In a related embodiment, the protein of interest is a recombinant protein and is expressed from an expression vector.

In yet another aspect, an aforementioned method is provided wherein the at least one protein of interest is selected from the group consisting of a nuclear lamina protein, a nucleolar protein, a transcription factor, a histone or histone variant, centromere protein A, a modification-specific internal antibody (mintbody), an intracellular scFV, a chromatin-modifying enzyme, an RNA polymerase, a DNA polymerase, a DNA helicase, a DNA repair protein, a Cas9 protein, a dCas9 protein, a zinc finger protein, a TALE protein, a CTCF protein, a cohesion protein, a synaptonemal complex protein, a telomere-binding protein, a centromere-binding protein, and an outer kinetochore protein. In still another aspect, an aforementioned method is provided wherein the at least one protein of interest has been engineered to modify one or more nucleotides at or near the DNA sequence.

In still another aspect, an aforementioned method is provided wherein contacting the DNA sequence by the at least one protein of interest results in a modification to the DNA that is detectable by imaging. In one embodiment, the modification is methylation. In another aspect or embodiment, the methylation occurs at or near a sequence comprising GATC. In still another aspect, an aforementioned method is provided wherein the protein of interest is a fusion of the protein of interest and (i) DNA adenine methyltransferase (Dam) or a biologically active fragment thereof, or (ii) EcoGII methyltransferase or a biologically active fragment thereof.

In another aspect of the present disclosure, an aforementioned method is provided wherein the collection of cells that expresses the at least one protein of interest also expresses at least one imaging protein that binds to methylation sites. In one aspect, the imaging protein is a fusion of a protein that binds methylated DNA and a green fluorescent protein (GFP) or a biologically active fragment thereof. In another aspect, the imaging protein is m6a-Tracer or m6A-Tracer-NES.

In various aspects, the cell is a bacterial cell, a eukaryotic cell or prokaryotic cell. In one aspect, the cell is a mammalian cell. In one aspect, the cell is a human cell.

In still another aspect, an aforementioned method is provided wherein the cellular location of the DNA sequence contacted by the protein of interest is determined by a method selected from the group consisting of microscopy, confocal microscopy, confocal fluorescent microscopy, high resolution microscopy, scanning confocal microscopy, two-photon fluorescence microscopy, TIRF microscopy, lattice light-sheet microscopy, super-resolution microscopy, and Stochastic Optical Reconstruction Microscopy.

In yet another aspect, an aforementioned method is provided wherein the amplifying the DNA sequence of part (c) comprises the steps of (i) lysing the single cell, (ii) digesting DNA, (iii) ligating universal primers, and (iv) PCR amplification. In one aspect, each step (i) - (iv) is performed in a separate chamber. In still another aspect, the lysing step comprises contacting the cell with a cell lysing agent selected from the group consisting of ionic and non-ionic detergents, Triton X-100, sodium dodecyl sulfate (SDS), NP-40, and ammonium chloride potassium. In yet another aspect, the digesting step comprises contacting the DNA from the lysed cell with a digesting agent selected from the group consisting of methyladenine-sensitive endoculease DpnI and methyladenine-sensitive endoculease DpnII. In one related aspect, the agent is DpnIor a biologically active fragment thereof.

In yet another aspect of the present disclosure, an aforementioned method is provided wherein the determining the sequence of the DNA sequence of step (d) allows the identification of an associated gene and/or locus within a genome.

In still another aspect, an aforementioned method is provided wherein the microfluidic device comprises from 1-100 lanes. In one aspect, each lane of the microfluidic device can carry out steps (b)-(c) in parallel.

In another aspect, a method of co-determining the cellular location and nucleotide sequence of a DNA that is contacted by a protein of interest in a single cell is provided, said method comprising the steps of: (a) incubating a collection of cells that express a protein of interest under conditions that allow the protein of interest to contact a DNA sequence comprising a DNA-binding site; (b) isolating a single cell from the collection of cells and determining the cellular location of the DNA comprising the DNA-binding site within the single cell; (c) amplifying and collecting the DNA comprising the DNA-binding site; and (d) determining the sequence of the DNA-binding site contacted by the protein of interest; wherein steps (b)-(c) are carried out in separate chambers within one lane of a microfluidic device; wherein the protein of interest is a fusion of the protein of interest and Dam or a biologically active fragment thereof; wherein the cellular location of step (b) is determined by confocal fluorescent microscopy; wherein the amplifying the DNA sequence of part (c) comprises the steps of (i) lysing the single cell, (ii) digesting DNA, (iii) ligating universal primers, and (iv) PCR amplification; and wherein the cellular location of the DNA comprising the DNA-binding site of step (b) is coupled to the sequence of the DNA-binding site of step (d) to provide contemporaneous imaging and sequence measurement of a protein-DNA interaction.

### BRIEF DESCRIPTION OF THE DRAWINGS

FIGS. 1A-1C show an µDamID device design and function. FIG. 1A: overview of DamID (van Steensel, B., & Henikoff, S., Nature Biotechnology, 18(4), 424-428, 2000) and ^{m6}A-Tracer (Kind et al., Cell, 153(1), 178-192, 2013) technologies applied to study interactions between DNA and nuclear lamina proteins. FIG. 1B: the overall design of a 10-cell device, showing the flow layer and the control layer. FIG. 1C: a closer view of one lane explaining the DamID protocol and the function of each chamber of the device. 10 cells are trapped, imaged, and selected serially, one per lane, then all 10 cells are processed in parallel.

FIG. 2 shows one embodiment of a cell trapping procedure. Cells are driven through the device by peristaltic pumping or pressure-driven flow. Valves are actuated to confine the cell in the trapping region, where it is imaged, and if selected, is pushed by dead-end filling into a holding chamber to the right of the trapping region.

FIGS. 3A-3F show the validation of µDamID sequencing data. FIG. 3A: comparison of bulk DamID sequencing data and aggregate single-cell sequencing data across all of human chromosome 1. log₂ ratios represent the ratio of Dam-LMNB1 sequencing coverage to normalized bulk Dam-only sequencing coverage. Positive values represent regions associated with the nuclear lamina, which tend to have lower gene density (second track from top). The Pearson correlation between bulk and aggregate single-cell data across all 250-kb bins in the genome is 0.85. FIG. 3B: scatterplot comparing raw sequencing coverage in bulk and single cell samples (aggregated). FIG. 3C: normalized coverage distribution in one single cell expressing Dam-LMNB1 (cell #8) in positive and negative control sets (cLADs, and ciLADs). The threshold that distinguishes these sets with maximal accuracy is shown as a dotted line. FIG. 3D: The maximum control set classification accuracy for each of 11 Dam-LMNB1 cells versus the number of unique DpnIfragments sequenced for each cell. Cell #8, the sample with median accuracy plotted in c, is labeled. FIG. 3E: Receiver-Operator Characteristic curves for all 11 cells, colored by the number of unique DpnIfragments sequenced. FIG. 3F: pairwise Pearson correlation heatmap for raw sequencing coverage in 250 kb bins genome-wide, with dendrogram indicating hierarchical clustering results. Numbers indicate cell numbers. DL = Dam-LMNB1. DO = Dam-only. Genes = number of Refseq genes in each bin. Kind = aggregated single-cell data from Kind et al. 2015. Bulk = bulk HEK293T DamID data from this study. *anomalous Dam-LMNB1 cell (#7) with high ^{m6}A-tracer signal in the nuclear interior

FIGS. 4A-4D show the results for ddefining variable LADs in HEK293T cells. FIG. 4A: A browser screenshot from Chr18:21-33 Mb. The first track shows the chromosome ideogram and coordinates. The second track reports the number of Refseq genes falling in each bin. The third track reports the mean Transcripts Per Million (TPM) value for each gene within each bin from bulk RNA-seq data from untreated HEK293T cells. The fourth track reports the log₂FoldChange values from bulk Dam-LMNB1:Dam-on1y sequencing data. The fifth track indicates the positions of the control cLAD and ciLAD sets as well as the positions of regions called as vLADs using the single-cell sequencing data generated here. The sixth track shows the number of single cells (out of 11) in which each bin is called as an LAD. Below that, the positions of all bins called as LADs are indicated, with one row per cell. FIG. 4B: Distribution of the number of single cells (out of 11) in which each bin is called as an LAD for all 250 kb bins genome-wide, separately for each of the control sets of cLADs or ciLADs. FIGS. 4C-D: Distributions of the number of genes or mean TPM per gene per 250 kb bin for each of the sets of cLADs, ciLADs, or vLADs.

FIGS. 5A-5D show joint imaging and sequencing analysis with µDamID. FIG. 5A: Confocal fluorescence microscopy images of ^{m6}A-Tracer GFP signal from 3 cells: one expressing Dam-only, one expressing Dam-LMNB1 but showing high interior fluorescence, and one expressing Dam-LMNB1 and showing the expected ring-like fluorescence at the nuclear lamina. FIG. 5B: Normalized pixel intensity values plotted as a function of their distance from the nuclear edge, with a fitted loess curve overlaid. Ratios of the mean normalized pixel intensities in the Lamina (<1 micron from the edge) versus the Interior (>3.5 microns from the edge) are printed on each plot. FIG. 5C: DamID sequencing coverage distributions for each of the cLAD or ciLAD control sets (as in FIG. 3C). FIG. 5D: scatterplot showing sequencing versus imaging metrics for each cell, with point size indicating the number of unique DpnIfragments sequenced for that cell. The x axis reports the log₂ ratio of the Lamina:Interior mean intensity ratio for each cell. The y axis reports the log₂ of the Signal-to-Noise Ratio (SNR) computed from the sequencing data for each cell (effectively the difference in means between cLADs and ciLADs divided by the standard deviation of ciLAD coverage).

FIGS. 6A-6D illustrate the improved signal-to-noise provided by m6A-Tracer with a C-terminal HIV-1 Rev Nuclear Export Signal (m6A-Tracer-NES) FIG. 6A: Illustration of potential mechanism by which m6A-Tracer-NES (m6A-Tracer with a C-terminal HIV-1 Rev Nuclear Export Signal) reduces background fluorescence in the nucleus caused by non-specific DNA interactions, due to the relative rates of export, diffusion, and DNA binding (indicated by horizontal arrows). FIG. 6B: Confocal fluorescent microscope images revealing the different localization patterns of m6A-Tracer (Kind et al., 2013) with/without a NES, and with/without Dam or Dam-LMNB1 co-expression. FIG. 6C: Confocal microscope images showing the localization of m6A-Tracer fluorescence when fused to one of two different Nuclear Export Signals on either terminus, in cells not expressing Dam. The HIV-1 Rev NES worked on either terminus and the C-terminal fusion was selected for downstream experiments. FIG. 6D: Time -lapse confocal microscope images of m6A-Tracer-NES fluorescence in the same field of cells at timepoints after Dam-LMNB1 expression. An inverted lookup table is used, and an arrow points to the nucleus of the same cell, which begins to show laminar signal around 2 h post-induction.

### DETAILED DESCRIPTION

Most approaches for mapping protein-DNA interactions rely on immunoaffinity purification, in which protein-DNA complexes are physically isolated using a high-affinity antibody against the protein, then purified by washing and decomplexed so the DNA can be amplified and sequenced. The most widely used among these methods is chromatin immunoprecipitation with sequencing (ChIP-seq; Johnson et al., Science, 316(5830), 1497-1502, 2007), which has formed the backbone of several large epigenome mapping projects (Celniker et al., Nature 459: 927-930 (2009)); ENCODE Consortium, Nature. 489: 57-74, 2012; Kundaje et al., Nature 518, 317-330 (2015)). One drawback of ChIP approaches is that the (often fragile) protein-DNA complex must survive through the shearing or digestion of the surrounding DNA, as well as through several intermediate washing and purification steps, in order to be amplified and sequenced. This results in a loss of sensitivity, especially when using a small amount of starting material. More recent methods have reduced the high input requirements of ChIP, but they still suffer from low sensitivity within single cells (Rotem et al., Nature Biotechnology, 33(11), 1165-U91, 2015; Harada et al., Nature Cell Biology, 1, 2018; Kaya-Okur et al., Nature Communications, 10(1), 1930, 2019).

An alternative method for probing protein-DNA interactions, called DNA Adenine Methyltransferase identification with high-throughput sequencing (DamID-seq), relies not on physical separation of protein-DNA complexes (as in ChIP-seq), but on a ‘chemical recording’ of protein-DNA interactions onto the DNA itself, which can later be selectively amplified (van Steensel et al. 2000). This method relies on a small enzyme from E. coli called DNA Adenine Methyltransferase (Dam). When genetically fused to the protein of interest, Dam deposits methyl groups at the N6 positions of adenine bases (m6A) within GATC sequences near the protein (which occur once every 270 bp on average across the human genome). That is, wherever the protein contacts DNA throughout the genome, m6A marks are left at GATC sites in its trail. These m6A marks are highly stable in eukaryotic cells, which do not tend to methylate adenines. Dam expression has been shown to have no discernable effect on gene expression in a human cell line, and its m6A marks were shown to be stably passed to daughter cells, halving in quantity each generation after Dam is inactivated (Park et al., Cell, 1-33, 2018). These properties allow even transient protein-DNA interactions to be recorded as biologically orthogonal and highly stable chemical signals on the DNA.

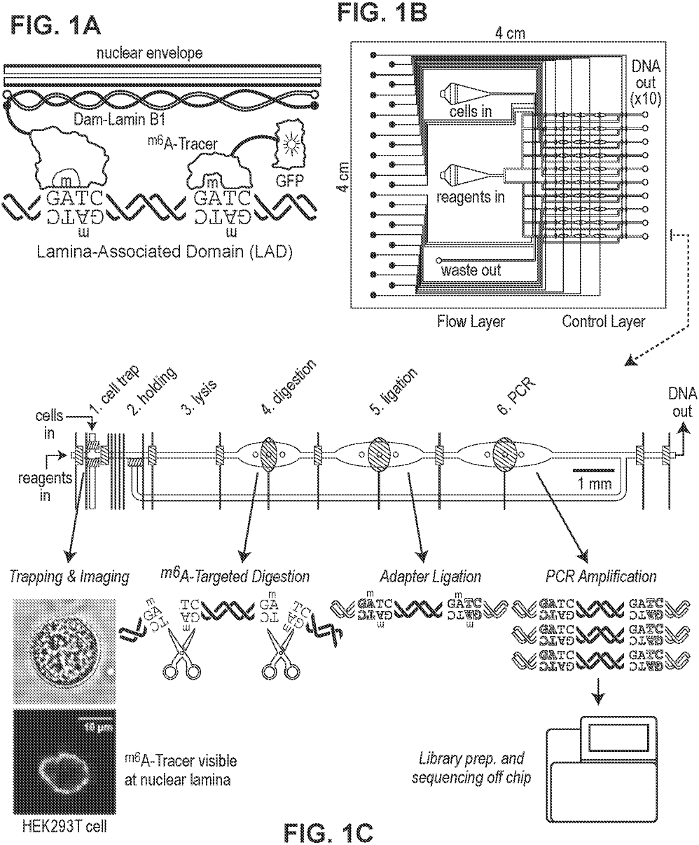

DamID reads out these chemical recordings of protein-DNA interactions by specifically amplifying and then sequencing fragments of DNA containing the interaction site. First, genomic DNA is purified and digested with DpnI, a restriction enzyme that exclusively cleaves G ^{m6} ATC sites (see, e.g., FIGS. 1A and 1C). Then, universal adapters are ligated onto the fragment ends to allow for amplification using universal primers. Only regions with a high density of ^{m6}A produce DNA fragments short enough to be amplified by Polymerase Chain Reaction (PCR) and quantified by microarray or high-throughput sequencing (Wu et al., JoVE (Journal of Visualized Experiments), (107), e53620-e53620, 2016). DamID has been used to explore dynamic regulatory protein-DNA interactions such as transcription factor binding (Orian et al., Genes & Development, 17(9), 1101-1114, 2003) and RNA polymerase binding (Southall et al., Developmental Cell, 26(1), 101-112, 2013) as well as protein-DNA interactions that maintain large-scale genome organization. One frequent application of DamID is to study large DNA domains associated with proteins at the nuclear lamina, near the inner membrane of the nuclear envelope (Pickersgill et al., Nature Genetics, 38(9), 1005-1014, 2006; Guelen et al., Nature, 453(7197), 948-951, 2008; and van Steensel and Belmont, Cell, 169(5), 780-791, 2017). Because DamID avoids the limitations of antibody binding, physical separations, or intermediate purification steps, it lends itself to single-cell applications. Recently, DamID has been successfully applied to sequence lamina-associated domains (LADs) in single cells in a one-pot reaction, recovering hundreds of thousands of unique fragments per cell (Kind et al., Cell. 163: 134-147,2015).

While DamID maps the sequence positions of protein-DNA interactions throughout the genome, the spatial location of these interactions in the nucleus can play an important role in genome regulation (Bickmore and van Steensel, Cell, 152(6), 1270-1284, 2013). A recent technique demonstrated the ability to specifically label and visualize protein-DNA interactions using fluorescence microscopy, revealing their spatial location within the nucleus in live cells (Kind et al. 2013). Visualization requires co-expression of a different fusion protein called ^{m6} A-Tracer, which contains green fluorescent protein (GFP) and a domain that binds specifically to methylated GATC sites. This imaging technology has been applied to visualize the dynamics of LADs within single cells (Kind et al. 2013). Many recent efforts have aimed to measure chromatin organization in single cells, to better understand the heterogeneity of cells within tissues and the biological underpinnings of their gene expression states (reviewed by Kelsey et al., Science, 358(6359), 69-75, 2017). Both imaging and sequencing protein-DNA interactions can provide useful single-cell epigenomic information, but despite recent advances in single-cell sequencing technologies, it remains fundamentally difficult to track individual cells and pair their sequencing data with other measurements such as imaging. Pairing imaging and sequencing data could be applied to study, for example, how the dynamic remodeling of chromatin proteins across the genome in developing cells relates to the localization of those proteins in the nucleus.

In some embodiments, the present disclosure pairs DamID sequencing with mTracer imaging to produce coupled imaging and sequencing measurements of protein-DNA interactions in the same single cells. This capability affords the user the ability to measure (and/or perturb) complex and dynamic cell processes in live cells under the microscope before taking an endpoint measurement to read out the chemical recordings of those protein-DNA interactions. This technology could be applied to study, in some embodiments, how the dynamic remodeling of chromatin proteins across the genome in developing cells relates to the localization of those proteins in the nucleus. While recent advances in single-cell sequencing methods allow for high-throughput isolation and processing of single cells, it remains fundamentally difficult to track individual cells or pair their sequencing data with other measurements. To address this, the present disclosure provides, in some embodiments, an integrated microfluidic device that enables single-cell isolation, imaging, selection, and DamID (referred herein as “µDamID”).

The present disclosure addresses the aforementioned unmet need by providing methods and materials for imaging and sequencing protein-DNA interactions in single cells.

### Definitions

The terms “polynucleotide” and “nucleic acid” refer to a polymer composed of a multiplicity of nucleotide units (ribonucleotide or deoxyribonucleotide or related structural variants) linked via phosphodiester bonds. A polynucleotide or nucleic acid can be of substantially any length, typically from about six (6) nucleotides to about 10⁹ nucleotides or larger. Polynucleotides and nucleic acids include RNA, cDNA, genomic DNA. In particular, the polynucleotides and nucleic acids of the present invention refer to polynucleotides encoding a chromatin protein, a nucleotide modifying enzyme and/or fusion polypeptides of a chromatin protein and a nucleotide modifying enzyme, including mRNAs, DNAs, cDNAs, genomic DNA, and polynucleotides encoding fragments, derivatives and analogs thereof. Useful fragments and derivatives include those based on all possible codon choices for the same amino acid, and codon choices based on conservative amino acid substitutions. Useful derivatives further include those having at least 50% or at least 70% polynucleotide sequence identity, and more preferably 80%, still more preferably 90% sequence identity, to a native chromatin binding protein or to a nucleotide modifying enzyme.

The term “oligonucleotide” refers to a polynucleotide of from about six (6) to about one hundred (100) nucleotides or more in length. Thus, oligonucleotides are a subset of polynucleotides. Oligonucleotides can be synthesized manually, or on an automated oligonucleotide synthesizer (for example, those manufactured by Applied BioSystems (Foster City, CA)) according to specifications provided by the manufacturer or they can be the result of restriction enzyme digestion and fractionation.

The term “primer” as used herein refers to a polynucleotide, typically an oligonucleotide, whether occurring naturally, as in an enzyme digest, or whether produced synthetically, which acts as a point of initiation of polynucleotide synthesis when used under conditions in which a primer extension product is synthesized. A primer can be single- stranded or double-stranded.

The term “nucleic acid array” as used herein refers to a regular organization or grouping of nucleic acids of different sequences immobilized on a solid phase support at known locations. The nucleic acid can be an oligonucleotide, a polynucleotide, DNA, or RNA. The solid phase support can be silica, a polymeric material, glass, beads, chips, slides, or a membrane. The methods of the present invention are useful with both macro- and micro-arrays.

The term “protein” or “protein of interest” refers to a polymer of amino acid residues, wherein a protein may be a single molecule or may be a multi-molecular complex. The term, as used herein, can refer to a subunit in a multi-molecular complex, polypeptides, peptides, oligopeptides, of any size, structure, or function. It is generally understood that a peptide can be 2 to 100 amino acids in length, whereas a polypeptide can be more than 100 amino acids in length. A protein may also be a fragment of a naturally occurring protein or peptide. The term protein may also apply to amino acid polymers in which one or more amino acid residues is an artificial chemical analogue of a corresponding naturally occurring amino acid. A protein can be wild-type, recombinant, naturally occurring, or synthetic and may constitute all or part of a naturally-occurring, or non-naturally occurring polypeptide. The subunits and the protein of the protein complex can be the same or different. A protein can also be functional or non-functional.

Non-limiting examples of a protein or protein of interest include, without limitation, a nuclear lamina protein (e.g., LMNB1 and LMNA), a nucleolar protein (e.g., NPM1 and NCL), a transcription factor (e.g., NPAT and SOX9), a histone or histone variant (e.g., CENPA and H3K9ac), centromere protein A, a modification-specific internal antibody (mintbody) (e.g., H3K9ac mintbody and H4K20me1 mintbody), an intracellular scFV, a chromatin-modifying enzyme (e.g., PRDM9 and HDAC2), an RNA polymerase subunit or modifier (e.g., RPB1 and CDK9), a DNA polymerase subunit or modifier (e.g., POLB and POLA2), a DNA helicase (e.g., MCM2 and RECQ1), a DNA repair protein (e.g., RAD51 and FANCD2), a Cas9 protein, a dCas9 protein, a zinc finger protein (e.g., PRDM9 and ZNF212), an engineered TALE protein, a CTCF protein, a cohesion protein (e.g., RAD21 and SMC1A), a synaptonemal complex protein (e.g., SYCP1 and SYCP2), a telomere-binding protein (e.g., TRF1 and TRF2), a centromere-binding protein (e.g., CENPC and CENPT), and an outer kinetochore protein(e.g., SPC24 and SPC25).

The term “chromatin” as used herein refers to a complex of DNA and protein, both in vitro and in vivo. This includes all proteins that are directly contacting DNA, and also proteins that are part of a protein or ribonucleoprotein complex that may be associated with DNA. A chromatin protein may or may not directly contact DNA. Chromatin also includes proteins that are transiently associated with DNA, with DNA-protein, or with DNA- ribonucleoprotein complexes, i.e., only during part of the cell cycle. “Chromatin protein” includes, but is not limited to histones, transcriptional factors, centromere proteins, heterochromatin proteins, euchromatin proteins, condensins, cohesins, origin recognition complexes, histone kinases, dephosphorylases, acetyltransferases, deacetylases, methyltransferases, demethylases, and other enzymes that covalently modify histone, DNA repair proteins, proteins involved in DNA replication, proteins involved in transcription, proteins part of dosage compensation complexes and X- chromosome inactivation, proteins that are part of chromatin remodeling complexes, telomeric proteins, and the like.

“Protein of interest- enzyme fusion polypeptide: or “chromatin protein-enzyme fusion polypeptide” refers to a polypeptide encoded by a polynucleotide encoding the chromatin protein operatively associated with a polynucleotide which encodes a nucleotide modification enzyme. Also encompassed within this definition are polynucleotides which encode a functionally active fragment, derivative or analog of the chromatin protein or nucleotide modification enzyme.

The term “polypeptide” refers to a polymer of amino acids and its equivalent and does not refer to a specific length of the product; thus, peptides, oligopeptides and proteins are included within the definition of a polypeptide. A “fragment” refers to a portion of a polypeptide having typically at least 10 contiguous amino acids, more typically at least 20, still more typically at least 50 contiguous amino acids of the chromatin protein. A “derivative” is a polypeptide which is identical or shares a defined percent identity with the wild-type chromatin protein or nucleotide modification enzyme. The derivative can have conservative amino acid substitutions, as compared with another sequence. Derivatives further include, for example, glycosylations, acetylations, phosphorylations, and the like. Further included within the definition of “polypeptide” are, for example, polypeptides containing one or more analogs of an amino acid (e.g., unnatural amino acids, and the like), polypeptides with substituted linkages as well as other modifications known in the art, both naturally and non-naturally occurring. Ordinarily, such polypeptides will be at least about 50% identical to the native chromatin binding protein or nucleotide modification enzyme acid sequence, typically in excess of about 90%, and more typically at least about 95% identical. The polypeptide can also be substantially identical as long as the fragment, derivative or analog displays similar functional activity and specificity as the wild-type chromatin protein or nucleotide modification enzyme.

The terms “amino acid” or “amino acid residue”, as used herein, refer to naturally occurring L amino acids or to D amino acids as described further below. The commonly used one- and three-letter abbreviations for amino acids are used herein (see, e.g., Alberts et al, Molecular Biology of the Cell, Garland Publishing, Inc., New York (3d ed. 1994)).

The term “isolated” refers to a nucleic acid or polypeptide that has been removed from its natural cellular environment. An isolated nucleic acid is typically at least partially purified from other cellular nucleic acids, polypeptides and other constituents.

“Functionally active polypeptide” refers to those fragments, derivatives and analogs displaying the functional activities associated with a full length protein of interest or chromatin protein or nucleotide modifying enzyme (e.g., binding the chromatin protein locus in the case of the fragments, derivatives of the protein of interest or chromatin protein and those fragments, derivatives and analogs of the nucleotide modifying enzyme which are capable of modifying a nucleotide in the case of the nucleotide modification enzyme, and the like).

The terms “identical” or “percent identity,” in the context of two or more nucleic acids or polypeptide sequences, refer to two or more sequences or subsequences that are the same or have a specified percentage of nucleotides or amino acid residues that are the same, when compared and aligned for maximum correspondence, as measured using one of the following sequence comparison algorithms, or by visual inspection.

The phrase “substantially identical,” in the context of two nucleic acids or polypeptides, refers to two or more sequences or subsequences that have at least 60%, typically 80%, most typically 90-95% nucleotide or amino acid residue identity, when compared and aligned for maximum correspondence, as measured using one of the following sequence comparison algorithms, or by visual inspection. An indication that two polypeptide sequences are “substantially identical” is that one polypeptide is immunologically reactive with antibodies raised against the second polypeptide.

“Similarity” or “percent similarity” in the context of two or polypeptide sequences, refer to two or more sequences or subsequences that are the same or have a specified percentage of amino acid residues or conservative substitutions thereof, that are the same, when compared and aligned for maximum correspondence, as measured using one of the following sequence comparison algorithms, or by visual inspection. By way of example, a first amino acid sequence can be considered similar to a second amino acid sequence when the first amino acid sequence is at least 30%, 40%, 50%, 60%, 70%, 75%, 80%, 90%, or even 95% identical, or conservatively substituted, to the second amino acid sequence when compared to an equal number of amino acids as the number contained in the first sequence, or when compared to an alignment of polypeptides that has been aligned by a computer similarity program known in the art, as discussed below. The term “substantial similarity” in the context of polypeptide sequences, indicates that the polypeptide comprises a sequence with at least 70% sequence identity to a reference sequence, or preferably 80%, or more preferably 85% sequence identity to the reference sequence, or most preferably 90% identity over a comparison window of about 10- 20 amino acid residues. In the context of amino acid sequences, “substantial similarity” further includes conservative substitutions of amino acids. Thus, a polypeptide is substantially similar to a second polypeptide, for example, where the two peptides differ only by one or more conservative substitutions.

The term “conservative substitution,” when describing a polypeptide, refers to a change in the amino acid composition of the polypeptide that does not substantially alter the polypeptide’s activity. Thus, a “conservative substitution” of a particular amino acid sequence refers to substitution of those amino acids that are not critical for polypeptide activity or substitution of amino acids with other amino acids having similar properties (e.g., acidic, basic, positively or negatively charged, polar or non-polar, and the like) such that the substitution of even critical amino acids does not substantially alter activity. Conservative substitution tables providing functionally similar amino acids are well known in the art. For example, the following six groups each contain amino acids that are conservative substitutions for one another: 1) alanine (A), serine (S), threonine (T); 2) aspartic acid (D), glutamic acid (E); 3) asparagine (N), glutamine (Q); 4) arginine (R), lysine (K); 5) isoleucine (I), leucine (L), methionine (M), valine (V); and 6) phenylalanine (F), tyrosine (Y), tryptophan (W). (See also Creighton, Proteins, W. H. Freeman and Company (1984).) In addition, individual substitutions, deletions or additions that alter, add or delete a single amino acid or a small percentage of amino acids in an encoded sequence are also “conservative substitutions.” For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. Optimal alignment of sequences for comparison can be conducted, for example, by the local homology algorithm of Smith & Waterman (Adv. Appl. Math. 2:482 (1981), which is incorporated by reference herein), by the homology alignment algorithm of Needleman & Wunsch (J. Mol. Biol. 48:443-53 (1970), which is incorporated by reference herein), by the search for similarity method of Pearson & Lipman (Proc. Natl. Acad. Sci. USA 85:2444-48 (1988), which is incorporated by reference herein), by computerized implementations of these algorithms (e.g., GAP, BESTFIT, FASTA, and TFASTA in the Wisconsin Genetics Software Package, Genetics Computer Group, 575 Science Dr., Madison, WI), or by visual inspection. (See generally Ausubel et al. (eds.), Current Protocols in Molecular Biology, John Wiley and Sons, New York (1996)).

One example of a useful algorithm is PILEUP. PILEUP creates a multiple sequence alignment from a group of related sequences using progressive, pairwise alignments to show the percent sequence identity. It also plots a tree or dendrogram showing the clustering relationships used to create the alignment. PILEUP uses a simplification of the progressive alignment method of Feng and Doolittle (J. Mol. Evol. 25:351-60 (1987), which is incorporated by reference herein). The method used is similar to the method described by Higgins & Sharp (Comput. Appl. Biosci. 5:151-53 (1989), which is incorporated by reference herein). The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. For example, a reference sequence can be compared to other test sequences to determine the percent sequence identity relationship using the following parameters: default gap weight (3.00), default gap length weight (0.10), and weighted end gaps.

Another example of algorithm that is suitable for determining percent sequence identity and sequence similarity is the BLAST algorithm, which is described by Altschul et al. (J. Mol. Biol. 215:403-410 (1990), which is incorporated by reference herein). (See also Zhang et al, Nucleic Acid Res. 26:3986-90 (1998); Altschul et al, Nucleic Acid Res. 25:3389-402 (1997), which are incorporated by reference herein). Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990), supra). These initial neighborhood word hits act as seeds for initiating searches to find longer HSPs containing them. The word hits are then extended in both directions along each sequence for as far as the cumulative alignment score can be increased. Extension of the word hits in each direction is halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLAST program uses as defaults a word length (W) of 11, the BLOSUM62 scoring matrix (see Henikoff & Henikoff, Proc. Natl. Acad. Sci. USA 89:10915-9 (1992), which is incorporated by reference herein) alignments (B) of 50, expectation (E) of 10, M=5, N=-4, and a comparison of both strands.

In addition to calculating percent sequence identity, the BLAST algorithm also performs a statistical analysis of the similarity between two sequences (see, e.g., Karlin & Altschul, Proc. Natl. Acad. Sci. USA 90:5873-77 (1993), which is incorporated by reference herein). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more typically less than about 0.01, and most typically less than about 0.001. Further, a polypeptide is typically substantially identical to a second polypeptide, for example, where the two peptides differ only by conservative substitutions. The terms “transformation” or “transfection” means a process of stably or transiently altering the genotype of a recipient cell or microorganism by the introduction of polynucleotides. This is typically detected by a change in the phenotype of the recipient cell or organism. The term “transformation” is generally applied to microorganisms, while “transfection” is used to describe this process in cells derived from multicellular organisms.

Generally, other nomenclature used herein and many of the laboratory procedures in cell culture, molecular genetics and nucleic acid chemistry and hybridization, which are described below, are those well-known and commonly employed in the art. (See generally Ausubel et al. (1996) supra; Sambrook et al, Molecular Cloning: A Laboratory Manual, Second Edition, Cold Spring Harbor Laboratory Press, New York (1989), which are incorporated by reference herein). Standard techniques are used for recombinant nucleic acid methods, polynucleotide synthesis, preparation of biological samples, preparation of cDNA fragments, isolation of mRNA and the like. Generally enzymatic reactions and purification steps are performed according to the manufacturers’ specifications.

The present disclosure provides methods and compositions for identifying the cellular location of proteins (e.g., “proteins of interest”) that directly or indirectly interact with a nucleic acid in a single cell, as well as the sequence of the aforementioned nucleic acid. In other aspects, the present disclosure provides methods and compositions for use in identifying the in vivo target loci of chromatin proteins in a single cell or in populations of living cells including, for example, specific tissues or cell populations isolated from an entire multicellular organism. More specifically, the methods and compositions comprise the use of the chromatin protein, or chromatin binding proteins, and chromatin binding fragments or derivatives thereof, linked or fused to an enzyme which modifies at least one, and typically more than one, nucleotide in the region associated with the target loci. In one specific example the modification enzyme is DNA adenine methyl transferase (Dam). Nucleotide sequences which have been modified are identified using, for example, an antibody specific for the modified nucleotide, restriction enzymes specific for particular modified nucleotide sequences, or by DNA micro-array methods. The technique, designated herein DamID (for DNA adenine methyl transferase IDentification), is sensitive and specific, and does not have any of the disadvantages of prior methods. As disclosed herein, the present disclosure is not limited to chromatin protein methods and compositions.

Chromatin, as above, is a complex of DNA and protein, e.g., in the nucleus of a cell in interphase. Many of these interactions require the presence of chromatin proteins which exert their regulatory and structural functions by binding to, or complexing with other proteins or nucleic acids, with a specific chromosomal loci. In the present invention the chromatin protein, or a specific binding fragment or derivative thereof is used to direct a nucleotide modification enzyme to the specific loci recognized by the chromatin protein. Any chromatin protein, or protein which recognizes a specific loci or sequence of nucleotides can be used to produce the fusion protein of the present invention. In specific embodiments of the present invention nucleotide sequences encoding Heterochromatin protein 1 (HP1), which binds predominantly to pericentric genes and transposable elements, GAGA factor (GF) which associates with euchromatic genes that are enriched in (GA)ₙ motifs, and a Drosophila homolog of the yeast Sir2 gene (DmSir2-1) which associates with certain active genes were used to construct exemplary fusion proteins of the invention.

A specific binding fragment or derivative of a protein of interest or chromatin protein comprises that portion of the protein of interest or chromatin protein or protein-nucleic acid complex required to recognize and bind the chromosomal loci or region recognized by the native protein of interest or chromatin protein. For example, a specific binding fragment of a Heterochromatin protein 1 (HP1), which binds predominantly to pericentric genes and transposable elements, GAGA factor (GF) which associates with euchromatic genes that are enriched in (GA)ₙ motifs, or a Drosophila homolog of the yeast Sir 2 gene (DmSir2-1) which associates with certain active genes can be used to construct a fusion protein of the invention. Fragments, derivatives or analogs of a protein of interest or chromatin protein or protein complex can be tested for the desired activity by procedures known in the art, including but not limited to the functional assays to determine whether the fragment recognizes and binds the target loci or nucleotide sequence recognized by the native full length chromatin binding protein. The affinity or avidity of the binding to the target loci or nucleotide sequence can be the same, less or greater than the affinity or avidity of the native full length protein. It is only necessary that the fragment, derivative or analog recognize and bind the target loci or sequence. In addition, the protein of interest or chromatin polypeptide fragment, derivative, or analog can be tested for the desired activity in the fusion protein to ensure localization to the appropriate loci.

Polypeptide derivatives include naturally-occurring amino acid sequence variants as well as those altered by substitution, addition or deletion of one or more amino acid residues that provide for functionally active molecules. Polypeptide derivatives include, but are not limited to, those containing as a primary amino acid sequence all or part of the amino acid sequence of a native protein of interest or chromatin polypeptide including altered sequences in which one or more functionally equivalent amino acid residues (e.g. , a conservative substitution) are substituted for residues within the sequence, resulting in a silent change.

In another aspect, polypeptides of the present invention include those peptides having one or more consensus amino acid sequences shared by all members of the protein of interest or chromatin protein family members, but not found in other proteins. Database analysis indicates that these consensus sequences are not found in other polypeptides, and therefore this evolutionary conservation reflects the nucleotide target binding-specific function of the protein of interest or chromatin polypeptides. Chromatin polypeptide family members, including fragments, derivatives and/or analogs comprising one or more of these consensus sequences, are also within the scope of the invention.

In another aspect, a polypeptide consisting of or comprising a fragment of a protein of interest or chromatin polypeptide having at least 5 contiguous amino acids of the protein of interest or chromatin polypeptide which recognize the specific target nucleotide sequence is provided. In other embodiments, the fragment consists of at least 20 or 50 contiguous amino acids of the protein of interest or chromatin polypeptide. In a specific embodiment, the fragments are not larger than 35, 100 or even 200 amino acids. Fragments, derivatives or analogs of chromatin polypeptide include, but are not limited to, those molecules comprising regions that are substantially similar to a chromatin polypeptide or fragments thereof (e.g., in various embodiments, at least 30%, 40%, 50%, 60%, 70%, 75%, 80%, 90%, or even 95% identity or similarity over an amino acid sequence of identical size), or when compared to an aligned sequence in which the alignment is done by a computer sequence comparison/alignment program known in the art, as described above, or whose coding nucleic acid is capable of hybridizing to a nucleic acid sequence encoding a protein of interest or chromatin protein, under high stringency, moderate stringency, or low stringency conditions. The choice of hybridization conditions will generally be guided by the purpose of the hybridization, the type of hybridization (DNA-DNA or DNA-RNA), and the level of relatedness between the sequences. Methods for hybridization are well established in the literature; See, for example: Sambrook, supra.; Hames and Higgins, eds, Nucleic Acid Hybridization A Practical Approach, IRL Press, Washington DC, (1985); Berger and Kimmel, eds, Methods in Enzymology, Vol. 52, Guide to Molecular Cloning Techniques, Academic Press Inc., New York, NY, (1987); and Bothwell et al, eds, Methods for Cloning and Analysis ofEukaryotic Genes, Jones and Bartlett Publishers, Boston, MA (1990); which are incorporated by reference herein in their entirety. The stability of nucleic acid duplexes will decrease with an increased number and location of mismatched bases; thus, the stringency of hybridization may be used to maximize or minimize the stability of such duplexes. Hybridization stringency can be altered by: adjusting the temperature of hybridization; adjusting the percentage of helix-destabilizing agents, such as formamide, in the hybridization mix; and adjusting the temperature and salt concentration of the wash solutions. In general, the stringency of hybridization is adjusted during the post- hybridization washes by varying the salt concentration and/or the temperature. Stringency of hybridization may be reduced by reducing the percentage of formamide in the hybridization solution or by decreasing the temperature of the wash solution. High stringency conditions involve high temperature hybridization (e.g., 65-68° C. in aqueous solution containing 4 to 6X SSC, or 42° C. in 50% formamide) combined with washes at high temperature (e.g., 5 to 25° C. below the Tₘ) at a low salt concentration (e.g., 0.1X SSC). Reduced stringency conditions involve lower hybridization temperatures (e.g., 35-42° C. in 20-50% formamide) with washes at intermediate temperature (e.g., 40 to 60° C.) and in a higher salt concentration (e.g., 2 to 6X SSC). Moderate stringency conditions involve hybridization at a temperature between 50° C. and 55° C. and washes in 0.1X SSC, 0.1% SDS at between 50° C. and 55° C. Nucleotide modifying enzymes, fragments, derivatives and analogs thereof useful in the present invention are those which can modify one or more nucleotides in a nucleic acid sequence, such as an RNA, DNA, or the like, under conditions found in a live cell and in a manner which is detectable. The enzyme must also modify the nucleotides in a manner which is not toxic to the cell. In other words, the cell or organism must be able to continue to proliferate and differentiate in a normal manner. For the modification to be detectable, an enzyme is selected which modifies the nucleotide in a manner which is not typical of a modification commonly found in the cell being assayed. For instance, in eukaryotic cells it is typical to select as the modification enzyme, for example, DNA adenine methyl transferase because methylation of adenine is not common in eukaryotic cells. Additional nucleotide modification enzymes useful in the present invention include, for example, but are not limited to, adenine methyltransferases, cytosine methyltransferases, thymidine hydroxylases, hydroxymethyluracil β-glucosyl transferases, adenosine deaminases, and the like. However, as described in more detail below, within one embodiment, a modification of the method of the present invention relies on an endogenous modification enzyme to modify DNA in a cell, the sites of such modifications are then determined by a variety of detection means, including the use of nucleic acid arrays.

In the methods of the present invention, the DNA modification enzyme, fragment, derivative, or analog thereof, is targeted to the loci associated with the binding of the protein of interest or chromatin protein by the protein of interest or chromatin protein, fragment, derivative or analog thereof, as a fusion protein. Typically, the polypeptides which comprise the protein of interest or chromatin protein and the DNA modification enzyme are separated from one another by one or more amino acid residues which comprise a linker sequence. The linker can be from about 1 to about 1000 amino acid residues, or more. Typically, the linker sequence is from about 3 to about 300 amino acid residues. The amino acid sequence can be from another polypeptide or can be an artificial sequence of amino acid residues, such as, for example, Gly and Ser residues which provide a flexible linear amino acid sequence allowing the amino acid sequences for the chromatin polypeptide and the nucleotide modification enzyme to fold into an active configuration. In a particular embodiment of the present invention a linker peptide comprising the myc-epitope tag GluGlnLysIleSerGluGluAspLeu EQKISEEDL (SEQ ID NO: 1) can be inserted between the protein of interest or chromatin polypeptide and the nucleotide modification enzyme DNA adenine methyl transferase.

The nucleotide sequence coding for a protein of interest or chromatin polypeptide -nucleotide modification enzyme fusion protein, or a functionally active derivative, analog or fragment thereof, can be inserted into an appropriate expression vector (i.e., a vector which contains the necessary elements for the transcription and translation of the inserted polypeptide -coding sequence). The necessary transcriptional and translational signals can also be supplied by a native gene and/or its flanking regions. A variety of vector systems can be utilized to express the polypeptide fusion-coding sequence. The choice of vector will be dependent on the cell to be transfected. The expression elements of vectors vary in their strengths and specificities. Depending on the cell- vector system utilized, any one of a number of suitable transcription and translation elements can be used. In specific embodiments, fusion proteins of the LMNB1 and CENPC fused with the nucleotide modification enzyme, E. coli DNA adenine methyl transferase, genes are expressed, or a nucleic acid sequence encoding a functionally active portion of the fusion proteins are expressed in, for example, Drosophila cells.

Any of the methods previously described for the insertion of DNA fragments into a vector can be used to construct expression vectors containing a chimeric gene consisting of appropriate transcriptional/translational control signals and the polypeptide coding sequences. These methods include in vitro recombinant DNA and synthetic techniques and in vivo recombinants (genetic recombination). Expression of a nucleic acid sequence encoding a fusion protein of the present invention or a fragment thereof can be regulated by a second nucleic acid sequence so that the fusion polypeptide or specific binding fragment is expressed in a host transformed with the recombinant DNA molecule. For example, expression of a fusion polypeptide can be controlled by any promoter/enhancer element known in the art. Promoters typically used in the present invention are those which provide low levels of expression of the fusion protein. Low levels of expression of the fusion protein are desired to avoid high background modification of non-targeted sequences. Suitable promoters can be selected empirically for each fusion protein by routine methods well known to the skilled artisan. Promoters suitable for use in the present invention include, but are not limited to, most heat shock promoters, for example, the hsp70 promoter, and various modified promoters, such as a truncated CMV promoter, and the like.

### Co-Determining the Cellular Location and Nucleotide Sequence of a DNA That Is Contacted by a Protein of Interest in a Single Cell

The present disclosure provides methods and materials for co-determining the cellular location and nucleotide sequence of a DNA that is contacted by (or in close proximity to) a protein of interest in a single cell. Thus the present disclosure provides methods and materials wherein the cellular location of the DNA comprising a DNA-binding site or otherwise in close proximity to a protein of interest is coupled to the sequence of said DNA to provide contemporaneous imaging and sequence measurement of a protein-DNA interaction.

As used herein, “contacted” as it relates to protein-DNA interactions includes direct contact or binding of a protein to a DNA at, for example, a DNA-binding site or sequence, and further includes indirect contact whereby a protein comes in sufficiently close proximity to a DNA sequence that allows a “mark” or other change to be imparted on the DNA sequence, as described herein.

In various embodiments, the protein of interest is a native protein, a wild-type protein, or a recombinant protein. In some embodiments, the protein is naturally-expressed by the cell or the cell is engineered to express, e.g., a recombinant protein, under specific conditions. In some embodiments, the “incubating” a collection of cells occurs in vitro. In other embodiments, a collection of cells is collected from a subject. In some embodiments, the cells are induced to express the protein of interest. In still another embodiment, the protein of interest is a recombinant protein and is expressed from an expression vector.

In some embodiments, the protein of interest is a fusion of the protein of interest and (i) DNA adenine methyltransferase (Dam) or a biologically active fragment thereof, or (ii) EcoGII methyltransferase or a biologically active fragment thereof. In other embodiments, the protein of interest is fused to Hia5, SSS1, CBIP1, TET1 or DNMT1.

In some embodiments, a collection of cells that express the at least one protein of interest also expresses at least one imaging protein that binds to DNA sites that have been contacted by the protein of interest. As one non-limiting example, in one embodiment at least one imaging protein binds to DNA methylation sites. In some embodiments, the imaging protein is a fusion of a protein that binds methylated DNA and a green fluorescent protein (GFP) or a biologically active fragment thereof, or a peptide tag such as a HaloTag that can covalently bind a fluorescent ligand. In some embodiments, the fusion complex can be delivered to the cell after fixing and permeabilizing the membrane of the cell.

In various embodiments, the cell is a bacterial cell, a eukaryotic cell, prokaryotic cell a mammalian cell, or a human cell. In some embodiments, the cell is a healthy cell or a diseased (e.g., cancer) cell or cell associated with a disease or disorder. In various embodiments, the cell is a lymphoblast, fibroblast, induced pluripotent stem cell, embryonic stem cell, adipocyte, or neural precursor cell.

### Microfluidic Devices

The present disclosure provides microfluidic devices which find use, for example, in the disclosed methods and systems. In some embodiments, a microfluidic device according to the present disclosure comprises at least one lane, wherein each lane comprises an inlet, an outlet, and a plurality of separate chambers. In various embodiments, the microfluidic device comprises, 1, 2, 3, 4, 5, 6, 7, 8, 9, 10, 15, 20, 25, 30, 35, 40, 45, 50, 55, 60, 65, 70, 75, 80, 85, 90, 95 or 100 lanes or more. In this way, single cells are captured and imaged in serial, and then they are all processed in parallel.

In one embodiment, an apparatus (e.g., a “microfluidic device”) is provided comprising a fluidics cartridge (e.g., chip or micro-chip) comprising at least one lane including an inlet, an outlet, and a plurality of separate chambers, the inlet adapted to receive a collection of cells, the collection of cells expressing at least one protein of interest under certain conditions that allow the at least one protein of interest to contact, or come in close to proximity to, a DNA sequence. In some embodiments, the apparatus further comprises a system comprising a cartridge receptacle adapted to receive the fluidics cartridge; a pump adapted to be in fluid communication with a reagent container containing a reagent and the inlet of the fluidics cartridge, the pump being configured to flow the reagent from the reagent container into the inlet of the fluidics cartridge to cause a single cell from the collection of cells to be isolated within one of the plurality of chambers of the fluidics cartridge; and an imaging assembly adapted to obtain image data of the single cell isolated within the one of the plurality of chambers of the fluidics cartridge.

In some embodiments, the at least one lane comprises separate chambers that allow (a) the injection of a collection of cells, (b) trapping of a single cell, (c) holding of the single cell, (d) lysis of the single cell, (e) digestion of the single cell, (f) ligation of primers to nucleic acid from the lysed single cell, (g) and amplification the nucleic acid. As described herein, the one or more lane further comprises inlets, outlets, and/or valves dispersed between one or more or all of the chambers. As described herein, the image data capture occurs, in some embodiments, while a single cell is in a cell trapping chamber. As described herein, each one or more lane further comprises in some embodiments an inlet to allow the injection of a reagent.

In some embodiments, a microfluidic device described herein further comprises a processor configured to access and process the image data to determine a cellular location of the DNA within the single cell.

In some embodiments, a microfluidic device described herein further comprises one or more valves adapted to constrain the single cell within the one of the plurality of chambers. In some embodiments, the valves are actuatable to flow the single cell from one chamber to another one of the plurality of chambers.

In some embodiments, a microfluidic device described herein further comprises a waste line coupled to the one of the plurality of chambers and adapted to selectively flow cellular debris to a waste reservoir.

In various embodiments, the isolation of a single cell, imaging of the single cell, and DNA amplification occurs in less than 1, 2, 3, 4, 5, 6, 7, 8, 9, or 10 minutes.

In some embodiments, a microfluidic device described herein is operably connected to an imaging device or image-capturing device. Exemplary image-capturing devices include, but are not limited to, a microscope, a confocal microscope, a confocal fluorescent microscope, a high resolution microscope, a scanning confocal microscope, a two-photon fluorescence microscope, a TIRF microscope, a lattice light-sheet microscope, a super-resolution microscope, and a stochastic optical reconstruction microscope.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “and,” and “the” include plural referents unless the context clearly dictates otherwise. Thus, for example, reference to “a conformation switching probe” includes a plurality of such conformation switching probes and reference to “the microfluidic device” includes reference to one or more microfluidic devices and equivalents thereof known to those skilled in the art, and so forth. It is further noted that the claims may be drafted to exclude any element, e.g., any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible. This is intended to provide support for all such combinations.

The following materials and methods were used in the Examples described herein.

### Cell Transfection and Harvesting

HEK293T cells (CRL-3216, ATCC, Manassas, VA; validated by microsatellite typing, at passage number <10) were seeded in 24-well plates at 50000 cells per well in 0.5 ml media (DMEM plus 10% FBS). The next day, cells were transfected using FuGene HD transfection reagent according to their standard protocol for HEK293 cells (Promega, Madison, WI). DNA plasmids were cloned in Dam-negative E. coli to reduce sequencing reads originating from plasmid. Dam-LMNB1 and ^{m6}A-Tracer plasmids were obtained from Bas van Steensel (from Kind et al. 2013); Dam-LMNB1 was modified to replace GFP with mCherry and to produce a Dam-only version; their sequences are available as supplementary information (see link to GitHub below in Data Availability section). 250 ng Dam construct DNA plus 250 ng ^{m6}A-Tracer DNA were used per well. As controls to validate transfection, additional wells were left untransfected, transfected with ^{m6}A-Tracer only, or transfected with Dam construct only. The following day, successful transfection was validated by widefield fluorescence microscopy, seeing GFP signal in wells containing ^{m6} A-Tracer, and mCherry signal in all wells containing Dam construct only. Cells were harvested 72 hours after transfection. 20 hours before harvesting, the media was replaced and 0.5 µl Shield-1 ligand (0.5 mM stock, Takara Bio USA, Inc., Mountain View, CA) was added to each well to stabilize protein expression. Cells transfected with Dam-LMNB1 were inspected by fluorescence microscopy to look for the characteristic signal at the nuclear lamina, indicating proper expression and protein activity. To harvest the cells and prepare them for loading on the device, the cells were washed with PBS, then incubated at room temperature with 1X TrypLE Select (ThermoFisher Scientific, Waltham, MA) for 5 minutes to dissociate them from the plate. Cells were pipetted up and down to break up clumps, then centrifuged at 300 xg for 5 minutes, resuspended in PBS, centrifuged again, and resuspended in 500 µl Pick Buffer (50 mM Tris-HCl pH 8.3, 75 mM KC1, 3 mM MgC12, 137 mM NaCl), achieving a final cell concentration of roughly 500,000 cells per ml. Cells were passed through a 40 µm cell strainer before loading onto the device.

### Confocal Imaging

Fluorescence confocal imaging of cells was performed in the trapping region using an inverted scanning confocal microscope with a 488 nm Ar/Kr laser (Leica, Germany) for excitation, with a bandpass filter capturing backscattered light from 500-540 nm at the primary photomultiplier tube (PMT), with the pinhole set to 1 Airy unit, with a transmission PMT capturing widefield unfiltered forward-scattered light, and with a 63X 0.7 NA long-working -distance air objective with a correction collar, zoomed by scanning 4X. Gain and offset values were set automatically for one cell and identical microscope settings were used to image all cells. The focal plane was positioned in the middle of each nucleus, capturing the largest-circumference cross-section, and final images were averaged over 10 frames to remove noise. The 3 cells expressing Dam-only that were sequenced in this study were imaged with a widefield CCD camera. Other Dam-only cells were imaged with confocal microscopy and showed similar relatively homogenous fluorescence throughout the nucleus, and never the distinct ‘ring’ shape found in Dam-LMNB1 expressing cells (Kind et al. 2013). No image enhancement methods were used prior to quantitative image processing.

### Mold Fabrication

Molds for casting each layer were fabricated on silicon wafers by standard photolithography. Photomasks for each layer were designed in AutoCAD and printed at 25400 DPI (CAD/Art Services, Inc., Bandon, Oregon). The mask for the thick layer, in this case the flow layer to make push-up valves, was scaled up in size uniformly by 1.5% to account for thick layer shrinkage. A darkfield mask was used for features made out of negative photoresist: the filters on the flow layer and the entire control layer; a brightfield mask was used for all flow layer channels, which were made out of positive photoresist (mask designs available on GitHub; see Data Availability section below). 10 cm diameter, 500 µm thick test-grade silicon wafers (item #452, University Wafer, Boston, MA) were cleaned by washing with 100% acetone, then 100% isopropanol, then DI water, followed by drying with an air gun, and heating at 200° C. for 5 minutes.

To make the control layer mold, SU-8 2025 negative photoresist (MicroChem Corp., Westborough, MA) was spin-coated to achieve 25 µm thickness (7 s at 500 rpm with 100 rpm/s ramp, then 30 s at 3500 rpm with 300 rpm/s ramp). All baking temperatures, baking times, exposure dosages, and development times followed the MicroChem data sheet. All baking steps were performed on pre-heated ceramic hotplates. After soft-baking, the wafer was exposed beneath the dark field control layer mask using a UV aligner (OAI, San Jose, CA). After post -exposure baking and development, the mold was hard-baked at 150° C. for 5 minutes.

To make the flow layer mold, first the filters were patterned with SU-8 2025, which was required to make fine, high-aspect-ratio filter features that would not re-flow at high temperatures. SU-8 2025 was spin-coated to achieve 40 µm thickness (as above but with 2000 rpm final speed) and processed according to the MicroChem datasheet as above, followed by an identical hard-bake step. Next, AZ 40XT-11D positive photoresist (Integrated Micro Materials, Argyle, TX) was spin-coated on top of the SU-8 features to achieve 20 µm thickness across the wafer (as above but with 3000 rpm final speed). All baking temperatures, baking times, exposure dosages, and development times followed the AZ 40XT-11D data sheet. After development, the channels were rounded by reflowing the photoresist, achieved by placing the wafer at 65° C. for 1 min, then 95° C. for 1 min, then 140° C. for 1 min followed by cooling at room temperature. In our experience, reflowing for too long, or attempting to hard-bake the AZ 40XT-11D resulted in undesirable ‘beading’ of the resist, especially at channel junctions. Because it was not hard-baked, no organic solvents were used to clean the resulting mold. Any undeveloped AZ 40XT-11D trapped in the filter regions was carefully removed using 100% acetone applied locally with a cotton swab.

### Soft Lithography

Devices were fabricated by multilayer soft lithography (Unger et al. 2000). On-ratio 10:1 base:crosslinker RTV615A PDMS (Momentive Performance Materials, Inc., Waterford, NY) was used for both layers, and layer bonding was performed by partial curing, followed by alignment, then full curing (Lai et al. 2019). To prevent PDMS adhesion to the molds, the molds were silanized by exposure to trichloromethlysilane (Sigma-Aldrich, St. Louis, MO) vapor under vacuum for 20 min. PDMS base and crosslinker were thoroughly mixed by an overhead mixer for 2 minutes, then degassed under vacuum for 90 minutes. Degassed PDMS was spin-coated on the control layer mold (for the ‘thin layer’) to achieve a thickness of 55 µm (7 s at 500 rpm with 100 rpm/s ramp, then 60 s at 2000 rpm with 500 rpm/s ramp), then placed in a covered glass petri dish and baked for 10 minutes at 70° C. in a forced-air convection oven (Heratherm OMH60, Thermo Fisher Scientific, Waltham, MA) to achieve partial curing. The flow layer mold (for the ‘thick layer’) was placed in a covered glass petri dish lined with foil, and degassed PDMS was poured onto it to a depth of 5 mm. Any bubbles were removed by air gun or additional degassing under vacuum for 5 minutes, then the thick layer was baked for 19 minutes at 70° C. Holes were punched using a precision punch with a 0.69 mm punch tip (Accu-Punch MP10 with CR0420275N19R1 punch, Syneo, Angleton, TX). The thick layer was peeled off the mold, cut to the edges of the device, and aligned manually under a stereoscope on top of the thin layer, which was still on its mold. The layers were then fully cured and bonded together by baking at 70° C. for 120 min. After cooling, the devices were peeled off the mold, and the inlets on the thin layer were punched. The final devices were bonded to 1 mm thick glass slides, which were first cleaned by the same method as used for silicon wafers above, using oxygen plasma reactive ion etching (20 W for 23 s at 285 Pa pressure; Plasma Equipment Technical Services, Brentwood, CA), followed by heating at 100° C. on a ceramic hot plate for 5 minutes.

### Device and Control Hardware Setup

Devices were pneumatically controlled by a solenoid valve manifold (Pneumadyne, Plymouth, MN). Each three-way, normally open solenoid valve switched between a regulated and filtered pressure source inlet at 25 psi (172 kPa) or ambient pressure to close or open microfluidic valves, respectively. Solenoid valves were controlled by the KATARA control board and software (White and Streets 2018). Most operational steps were carried out on inverted microscopes using 4-10X objectives. For incubation steps, the device was placed on a custom-built liquid-cooled thermoelectric temperature control module (TC-36-25-RS232 PID controller with a 36 V 16 A power source and two serially connected VT-199-1.4-0.8P TE modules and an MP-3022 thermistor; TE technologies, Traverse City, MI) controlled by a new KATARA software module (to be made available on github). A layer of mineral oil was applied between the chip and the temperature controller to improve thermal conductivity and uniformity. A stereoscope was used to monitor the chip while on the temperature controller.

To set up each new device, each pneumatic valve was connected to one control inlet on the microfluidic device by serially connecting polyurethane tubing (3/32”ID, 5/32” OD; Pneumadyne) to Tygon tubing (0.5 mm ID, 1.5 mm OD) to >4 cm PEEK tubing (0.25 mm ID, 0.8 mm OD; IDEX Corporation, Lake Forest, IL). Solenoid valves were energized to depressurize the tubing and the tubing was primed by injecting water using a syringe connected to the end of the PEEK tubing, then the primed PEEK tubing was inserted directly into each punched inlet hole on the device. Solenoid valves were de-energized to pressurize the tubing until all control channels on the device were fully dead-end filled, then each microfluidic valve was tested and inspected by switching on and off its corresponding solenoid valve. All valves were opened and the device was passivated by filling all flow-layer channels with syringe -filtered 0.2% (w/w) Pluronic F-127 solution (P2443; MilliporeSigma, St. Louis, MO) from the reagent inlet and incubating at room temperature for 1 hour. The device was then washed by flowing through 0.5 ml of ultra-filtered water, followed by air to dry it.

### Device Operation

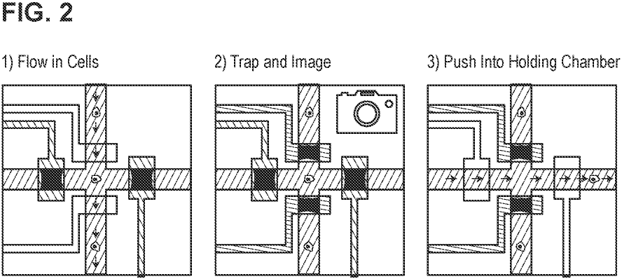

Initially, all chamber valves and reagent inlet valves were closed. Gel-loading pipette tips were used to inject reagents and cells into the flow channels. To prepare the device for operation, pick buffer was injected into the reagent inlet and pressurized at 5-10 psi to dead-end fill the reagent inlet channels. Negative controls were generated by injecting pure pick buffer into one of the holding chambers before trapping and sorting cells into the other lanes. 50 µl of cell suspension was then loaded into a gel-loading pipette tip, and injected directly into the cell inlet. A high-precision pressure regulator was used to load the single-cell suspension at 1 psi (7 kPa). Cells were observed in the filter region with brightfield and epifluorescence using a 10X objective to identify candidate cells. These were then tracked through the device until they approached the trapping chamber for an empty lane. To trap a candidate cell, the device’s peristaltic pump was operated at 1 Hz to deliver that cell to the trap region. The trap valves (above and below the trap region; see FIG. 2) were closed and the cell was imaged with scanning confocal microscopy as described above. If the cell was rejected after imaging, the trap valves were opened and it was flushed to the waste outlet. Otherwise, the cell was injected into the holding chamber by dead-end filling. This process was repeated to fill each lane with single cells for DamID. To test background DNA levels, we filled the final lane with only cell suspension buffer. Nearly undetectable levels of amplified DNA were recovered from these lanes.

After filling all 10 lanes, the reagent inlet and cell trapping channels were flushed with 0.5 ml of water, which exited through the waste outlet and the cell inlet, to remove any remaining Pick buffer or cell debris, then air dried. The same washing and drying was repeated between each reaction step. To inject reagents for each reaction of the DamID protocol, the trap valves were closed, the reagent channels were dead-end filled with freshly prepared and syringe -filtered reagent, then the reagent inlet valves and the valves for the necessary reaction chambers were opened, and each lane was dead-end filled individually to prevent any possible cross -contamination. Reaction contents are described in Table 1.

After filling all lanes, reagents were mixed by actuating the chamber valves at 5 Hz for 5 minutes. At the PCR step, rotary mixing was achieved by using the chamber valves to make a peristaltic pump driving fluid around the full reaction ring. For each reaction step, the device was placed on the thermal controller and reactions were with times and temperatures described in Table 1. PCR thermocycling conditions are described in Table 2. To ensure adequate hydration during PCR, all valves were pressurized. Amplified DNA was flushed out of each lane individually using purified water from the reagent inlet, collected into a gel loading tip placed in the lane outlet to a final volume of 5 µl then transferred to a 0.2 ml PCR strip tube.

TABLE 1

Reaction buffers and conditions

| | | |
|---|---|---|
| Reaction Stage | Buffer | Incubation |
| Trapping & Holding | Pick Buffer: 50 mM Tris-HCl pH 8.3 75 mM KCl, 3 mM MgCl₂ 137 mM NaCl | RT |
| Lysis | 10 mM TRIS acetate pH 7.5 10 mM magnesium acetate 50 mM potassium acetate 0.67% Tween-20 0.67% Igepal 0.67 mg/ml proteinase K | 42° C. for 4 hours then 80° C. for 10 min |
| Digestion | mix 7 µl 10X Cutsmart buffer 1 µl DpnI (New England Biolabs, Ipswich, MA) 62 µl H₂O | 37° C. for 4 hours then 80° C. for 20 min |
| Ligation | mix 6 µl 10X NEB T4 ligase buffer 1 µl DamID adapter stock at 25 µM 0.2 µl NEB T4 ligase at 400 U/µl 21.8 µl H₂O 1 µl 2% w/v Tween-20 | 16 °C overnight then 65° C. for 10 min |
| PCR | from Takara Clontech Advantage 2 kit: mix 5 µl 10X PCR buffer 1 µl dNTPs at 10 mM each 1 µl polymerase mix 0.63 µl DamID primer (100 µM) 21.37 µl H₂O 1 µl 2% Tween-20 | See Table 2 |

TABLE 2

PCR thermocycling conditions

| PCR Step | Incubation |
|---|---|
| 1 | 68° C. for 10 min |
| 2 | 94° C. for 1 min |
| 3 | 65° C. for 5 min |
| 4 | 68° C. for 15 min |
| 5 | 94° C. for 1 min |
| 6 | 65° C. for 1 min |
| 7 | 68° C. for 10 min |
| 8 | Go to step 5 (x 3) |
| 9 | 94° C. for 1 min |
| 10 | 65° C. for 1 min |
| 11 | 68° C. for 2 min |
| 12 | Go to step 9 (x 22) |
| 13 | Hold 10° C. |

Oligonucleotides

>AdRt
CTAATACGACTCACTATAGGGCAGCGTGGTCGCGGCCGAGGA (SEQ ID NO: 2)
>AdRb
TCCTCGGCCG (SEQ ID NO: 3)
>AdR_PCR
NNNNGTGGTCGCGGCCGAGGATC (SEQ ID NO: 4)

To anneal DamID adapter (Vogel et al., Nature Protocols, 2(6), 1467-1478, 2007): mix equal volumes of 50 µM AdRt and 50 µM AdRb in a microcentrifuge tube, then fully submerge it in a beaker of boiling water, and allow the water to equilibrate to room temperature slowly.

### Quality Control, Library Preparation, and Sequencing

Samples were diluted to 10 µl total volume and two replicates of qPCR were performed using the DamID PCR primer to estimate DNA quantities relative to the pick-buffer-only negative control (1 µl DNA per replicate in 10 µl reaction volume). We also used 1 µl of sample to measure DNA concentration using a Qubit fluorometer with a High-Sensitivity DNA reagent kit (quantitative range 0.2 ng - 100 ng; ThermoFisher Scientific). Samples with the lowest Ct values and highest Qubit DNA measurements were selected for library preparation and sequencing. Library preparation was carried out using an NEBNext Ultra II DNA Library Prep Kit for Illumina (NEB E7645) with dual-indexed multiplex i5/i7 oligo adapters. Size selection was not performed; PCR was carried out for 9 cycles. Libraries were quantified again by Qubit and size profiled on a TapeStation 4200 with a D5000 HS kit (Agilent, Santa Clara, CA), then mixed to achieve equimolar amounts of each library. DNA was sequenced on an Illumina MiniSeq with a 150-cycle high output kit, to produce paired 75 bp reads, according to manufacturer instructions (Illumina, San Diego, CA). Roughly 13 million read pairs were obtained.

### Bulk DamID

Genomic DNA was isolated from ∼3.7 × 10⁶ transfected HEK293T cells using the DNeasy Blood & Tissue kit (Qiagen) following the protocol for cultured animal cells with the addition of RNase A. The extracted gDNA was then precipitated by adding 2 volumes of 100% ethanol and 0.1 volume of 3 M sodium acetate (pH 5.5) and storing at -20° C. for 30 minutes. Next, centrifugation for 30 minutes at 4° C., >16,000 × g was performed to spin down the gDNA. The supernatant was removed, and the pellet was washed by adding 1 volume of 70% ethanol. Centrifugation for 5 minutes at 4° C., >16,000 × g was performed, the supernatant was removed, and the gDNA pellets were air-dried. The gDNA was dissolved in 10 mM Tris-HCl pH 7.5, 0.1 mM EDTA to 1 µg/µl, incubating at 55° C. for 30 minutes to facilitate dissolving. The concentration was measured using Nanodrop.

The following DpnI digestion, adaptor ligation, and DpnII digestion steps were all performed in the same tube. Overnight DpnI digestion at 37° C. was performed with 2.5 µg gDNA, 10 U DpnI (NEB), 1X CutSmart (NEB), and water to 10 µl total reaction volume. DpnI was then inactivated at 80° C. for 20 minutes. Adaptors were ligated by combining the 10 µl of DpnI-digested gDNA, 1X ligation buffer (NEB), 2 µM adaptor dsAdR, 5 U T4 ligase (NEB), and water for a total reaction volume of 20 µl. Ligation was performed for 2 hours at 16° C. and then T4 ligase was inactivated for 10 minutes at 65° C. DpnII digestion was performed by combining the 20 µl of ligated DNA, 10 U DpnII (NEB), 1X DpnII buffer (NEB), and water for a total reaction volume of 50 µl. The DpnII digestion was 1 hour at 37° C. followed by 20 minutes at 65° C. to inactivate DpnII.

Next, 10 µl of the DpnII-digested gDNA was amplified using the Clontech Advantage 2 PCR Kit with 1X SA PCR buffer, 1.25 µM Primer Adr-PCR, dNTP mix (0.2 mM each), 1X PCR advantage enzyme mix, and water for a total reaction volume of 50 µl. PCR was performed with an initial extension at 68° C. for 10 minutes; one cycle of 94° C. for 1 minute, 65° C. for 5 minutes, 68° C. for 15 minutes; 4 cycles of 94° C. for 1 minute, 65° C. for 1 minute, 68° C. for 10 minutes; 21 cycles of 94° C. for 1 minute, 65° C. for 1 minute, 68° C. for 2 minutes. Post -amplification DpnII digestion was performed by combining 40 µl of the PCR product with 20 U DpnII, 1X DpnII buffer, and water to a total volume of 100 µl. The DpnII digestion was performed for 2 hours at 37° C. followed by inactivation at 65° C. for 20 minutes. The digested product was purified using QIAquick PCR purification kit.

The purified PCR product (1 µg brought up to 50 µl in TE) was sheared to a target size of 200 bp using the Bioruptor Pico with 13 cycles with 30”/30” on/off cycle time. DNA library preparation of the sheared DNA was performed using NEBNext Ultra II DNA Library Prep Kit for Illumina.

### Bulk DamID, Comparing Dam Mutants

Bulk DamID for comparing the wild-type allele and V133A mutant allele was performed as outlined in the Bulk DamID methods section with the following modifications. Genomic DNA was extracted from ∼ 2.4 × 10⁵ transfected HEK293T cells. A cleanup before methylation -specific amplification was included to remove unligated Dam adapter before PCR. The Monarch PCR & DNA Cleanup Kit with 20 µl DpnII-digested gDNA input and an elution volume of 10 µl was used. Shearing with the Bioruptor Pico was performed for 20 total cycles with 30”/30” on/off cycle time. Paired-end 2 × 75 bp sequencing was performed on an Illumina NextSeq with a mid output kit. Approximately 3.8 million read pairs per sample were obtained.

### Bulk RNA-Seq

RNA was extracted from ~1.9 × 10⁶ transfected HEK293T cells using the RNeasy Mini Kit from Qiagen with the QIAshredder for homogenization. RNA library preparation was performed using the NEBNext Ultra II RNA Library Prep Kit for Illumina with the NEBNext Poly(A) mRNA Magnetic Isolation Module. Paired-end 2 × 150 bp sequencing for both DamID-seq and RNA-seq libraries was performed on 1 lane of a NovaSeq S4 run. Approximately 252 million read pairs were obtained for each DamID-seq sample, and roughly 64 million read pairs for each RNA sample. Adapters were trimmed using trimmomatic (v0.39; Bolger et al., Bioinformatics (Oxford, England), 30(15), 2114-2120, 2014; ILLUMINACLIP:adapters-PE.fa:2:30:10 LEADING:3 TRAILING:3 SLIDINGWINDOW:4:15 MINLEN:36, where adapters-PE.fa is:

>PrefixPE/1
TACACTCTTTCCCTACACGACGCTCTTCCGATCT (SEQ ID NO: 5)
>PrefixPE/2
GTGACTGGAGTTCAGACGTGTGCTCTTCCGATCT) (SEQ ID NO: 6)

Transcript quantification was performed using Salmon (Patro et al., Nature Methods, 14(4), 417-419, 2017) with the GRCh38 transcript reference. Differential expression analysis was performed using the voom function in limma (Ritchie et al., Nucleic Acids Research, 43(7), e47-e47, 2015). Differential expression was called based on logFC significantly greater than 1 and adjusted p-value < 0.01.

### DamID Sequence Processing and Analysis

Bulk and single-cell DamID reads were demultiplexed using Illumina’s BaseSpace platform to obtain fastq files for each sample. DamID and Illumina adapter sequences were trimmed off using trimmomatic (v0.39; Bolger et al. 2014; ILLUMINACLIP:adapters-PE.fa:2:30:10 LEADING:3 TRAILING:3 SLIDINGWINDOW:4:15 MINLEN:20, where adapters-PE.fa is:

>PrefixPE/1
TACACTCTTTCCCTACACGACGCTCTTCCGATCT (SEQ ID NO: 5)
>PrefixPE/2
GTGACTGGAGTTCAGACGTGTGCTCTTCCGATCT (SEQ ID NO: 6)
>Dam
GGTCGCGGCCGAGGA (SEQ ID NO: 7)
>Dam_rc
TCCTCGGCCGCGACC (SEQ ID NO: 8)

Trimmed reads were aligned to a custom reference (hg38 reference sequence plus the Dam-LMNB1 and ^{m6}A-Tracer plasmid sequences) using BWA-MEM (v0.7.15-r1140, Li, 2013, arXiv:1303.3997 [q-bio.GN]). Alignments with mapping quality 0 were discarded using samtools (v1.9, Li et al., Bioinformatics (Oxford, England), 25(16), 2078-2079, 2009). The hg38 reference sequence was split into simulated DpnI digestion fragments by reporting all intervals between GATC sites (excluding the GATC sites themselves), yielding 7180359 possible DpnI fragments across the 24 chromosome assemblies. The number of reads overlapping each fragment was counted using bedtools (v2.28; Quinlan et al., Bioinformatics (Oxford, England), 26(6), 841-842, 2010). For single-cell data, the number of DpnI fragments with non-zero coverage was reported within each non-overlapping bin in the genome (11512 total 250 kb bins). For bulk data, the number of read pairs overlapping each 250 kb bin was reported. The same exact pipeline was applied to the raw reads from Kind et al. 2015 in aggregate. RefSeq gene positions were downloaded from the UCSC Genome Browser and counted in each bin. For bulk data, Dam-LMNB1 vs DamOnly enrichment was computed using DEseq2 in each 250 kb bin (Love et al., Genome Biology, 15(12), 550, 2014). For single-cell data, the expected background coverage in each bin was computed as n(m/t), where n is the number of unique fragments sequenced from that cell, m is the number of bulk Dam-only read pairs mapping to that bin, and t is the total number of mapped bulk Dam-only read pairs. Single-cell normalization was computed either as a ratio of observed to expected coverage (for browser visualization and comparison to bulk data), or as their difference (for classification and coverage distribution plotting). Positive and negative control sets of cLAD and ciLAD bins were defined as those with a bulk Dam-LMNB 1:Dam-only DEseq2 p-value smaller than 0.01/11512, that intersected published cLADs and ciLADs in other cell lines (Lenain et al., Genome Res. 2017 Oct;27(10):1634-1644), and that were among the top 1200 most differentially enriched bins in either direction (positive or negative log fold change for cLADs and ciLADs, respectively). Integer normalized coverage thresholds for LAD/iLAD classification were computed for each cell to maximize accuracy on the cLAD and ciLAD control sets. Signal-to-noise ratios were computed for each cell using the normalized coverage distributions in the cLAD and ciLAD control sets as (µ_{cLAD} - µ_{ciLAD})/σ_{ciLAD}. Statistical analyses and plots were made in R (v3.5.2) using the ggplot2 (v3.1.0), gplots (v3.0.1.1), and colorRamps (v2.3) packages. Browser figures were generated using the WashU Epigenome Browser (Li et al., Nucleic Acids Res. 2019 Jan 8;47(D1):D983-D988).

### Image Processing

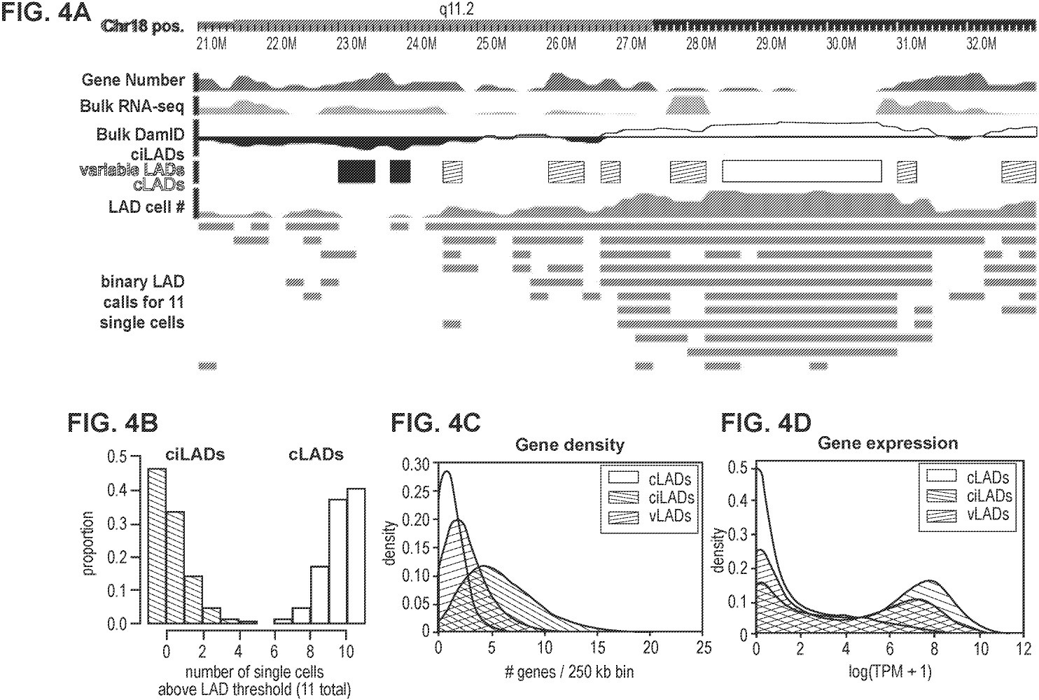

Images were processed in R (v3.5.2) and plots were produced using the reshape2 (v1.4.3), SDMTools (v1.1-221.1), spatstat (v1.59-0), magick (v2.0), ggplot2 (v3.1.0), and ggbeeswarm (v0.6) packages. Grayscale images were converted to numeric matrices and edge detection was performed using Canny edge detection using the image_canny function in magick, varying the geometry parameters manually for each cell. The center of mass of all edge points was obtained, and all edge points were plotted in Cartesian coordinated with this center of mass as the origin. Noise was removed by removing points with a nearest neighbor more than 2 microns away. Edge point coordinates were converted to polar coordinates, and the farthest points from the origin in each 10 degree arc were reported. Within each 10 degree arc, all pixel intensities from the original image within the edges of the nucleus were reported as a function of their distance from the farthest edge point in that arc to make FIG. 4B. For each cell a loess curve (span 0.3) was fitted to the data after subtracting the minimum intensity value within 3.5 microns of the edge. The Lamina:Interior ratio was computed as the ratio of mean intensity of pixels within 1 micron of the edge to the mean intensity of pixels more than 3.5 microns from the edge, after subtracting the minimum value of the loess curve for that cell.

### EXAMPLE 1

### Design and Manufacture of Microfluidic Device

This Example provides a design and fabrication of a microfluidic device according to one embodiment of the present disclosure.

A polydimethylsiloxane (PDMS) microfluidic device was designed and fabricated with integrated elastomeric valves to facilitate the various reaction stages of the DamID protocol in a single liquid phase within the same device (FIG. 1C). The device is compatible with high -magnification imaging on inverted microscopes, enabling imaging prior to cell lysis. Each device was designed to process 10 cells in parallel, each in an individual reaction lane fed from a common set of inlets. Valves are controlled by pneumatic actuators operated electronically via a programmable computer interface (White and Streets , HardwareX, 3, 135-145, 2018).

Device operation was modified from a single-cell RNA sequencing platform (Streets et al., PNAS May 13, 2014 111 (19) 7048-7053). A suspension of single cells is loaded into the cell inlet (FIG. 1B) and cells are directed towards a trapping region by a combination of pressure-driven flow and precise peristaltic pumping. As a cell crosses one of the 10 trapping regions, valves are actuated to immobilize the cell for imaging (FIG. 2). The cell is imaged by confocal fluorescence microscopy to visualize the localization of ^{m6} A-Tracer, and after image acquisition, the user can choose whether to select the cell for DamID processing, or to reject it and send it out the waste outlet (FIG. 1B).

Selected cells are injected from the trapping region into a holding chamber using pressure-driven flow from the reagent inlet (FIG. 1B, FIG. 2). Once all 10 holding chambers are filled with imaged cells, processing proceeds in parallel for all 10 cells by successively adding the necessary reagents for each step of the single-cell DamID protocol (Kind et al. 2015) and dead-end filling each of the subsequent reaction chambers. Reaction temperatures are controlled by placing the device on a custom-built thermoelectric control unit for dynamic thermal cycling. Enzymes are heat inactivated between each step (Kind et al. 2015) and a low concentration of mild detergent was added to all reactions to prevent enzyme adhesion to PDMS (Streets et al. 2014).

FIGS. 1A-1C shows a schematic of the microfluidic processing work flow. In the first reaction stage, a buffer containing detergent and proteinase pushes the cell into the lysis chamber, where its membranes are lysed and its proteins, including ^{m6} A-Tracer, are digested away. Next, a DpnI reaction mix is added to digest the genomic DNA at Dam-methylated GATC sites in the digestion chamber. Then, a mix of DamID universal adapter oligonucleotides and DNA ligase is added to the ligation chamber. Finally, a PCR mix is added containing primers that anneal to the universal adapters is added and all valves within the lane are opened, creating a 120 nl cyclic reaction chamber. Contents are thoroughly mixed by peristaltic pumping around the reaction ring, then PCR is carried out on-chip by thermocycling. Amplified DNA is collected from each individual lane outlet, and sequencing library preparation is carried out off -chip.

### EXAMPLE 2

### Mapping the Sequence and Spatial Location of Lamina-Associated Domains in a Human Cell Line

This Example describes the use of an integrated microfluidic device for single-cell isolation, imaging, and sorting, followed by DamID.

### A. Application of a Microfluidic Device to Map Lamina-Associated Domains in a Human Cell Line

The performance of platform described herein was evaluated by mapping the sequence and spatial location of lamina-associated domains in a human cell line, allowing comparison of data to previously published LAD maps from DamID experiments in human cell lines (Kind et al. 2015, Lenain et al. 2017). LADs are large (median 500 kb) and comprise up to 30% of the genome in human cells (Guelen et al., Nature, 453(7197), 948-951, 2008). LADs serve both a structural function, acting as a scaffold that underpins the three-dimensional architecture of the genome in the nucleus, and a regulatory function, as LADs tend to be gene-poor, more heterochromatic, and transcriptionally less active (reviewed by van Steensel and Belmont 2017 and Buchwalter et al., Nature Reviews Genetics, 1-12, 2018). ^{m6}A-Tracer has previously been applied to visualize LADs, which appear as a characteristic ring around the nuclear periphery in confocal fluorescence microscopy images (Kind et al. 2013; FIG. 1C).

HEK293T cells were used for their ease of growth, transfection, suspension, and isolation. To enable rapid expression of Dam and ^{m6}A-Tracer transgenes, cells were transiently transfected with DNA plasmids containing genes for a drug-inducible Dam-LMNB1 fusion protein as well as constitutively expressed ^{m6} A-Tracer. Dam-LMNB1 expression was then induced, optimizing the expression times to maximize the proportion of cells with fluorescent laminar rings (FIG. 1C). Because transient transfection yields a heterogeneous population of cells, each with potentially variable copies of the transgenes, it was important to be able to image cells and select only those with visible laminar rings, which were more likely to have the correct expression levels, and which were unlikely to be in the mitosis phase of the cell cycle. This kind of complex sorting would not be possible with sorting methods like fluorescence-activated cell sorting (FACS) but is straight-forward in our microfluidic platform.

In addition to processing Dam-LMNB1 cells, cells were transfected with the Dam gene alone, not fused to LMNB1, to provide a negative control demonstrating where the unfused Dam enzyme would mark the genome if not tethered to the nuclear lamina (Vogel et al., Nature Protocols, 2(6), 1467-1478, 2007). This control is useful for estimating the background propensity for each genomic region to be methylated, since Dam preferentially methylates more accessible regions of the genome, including gene-rich regions (Singh and Klar, Genes & Development, 6(2), 186-196, 1992, Lenain et al. 2017, Aughey et al., Wiley Interdisciplinary Reviews: Developmental Biology, 5(1), 25-37, 2018). Dam-only cells were selected that had high fluorescence levels across the nucleus and did not appear mitotic. DamID was also performed in bulk transiently transfected HEK293T cells for validation (Vogel et al. 2007). A mutant of Dam (V133A; Elsawy and Chahar 2014) was used, which is predicted to have weaker methylation activity than the wild-type allele on unmethylated DNA, to reduce background methylation. Bulk DamID experiments were performed comparing the mutant and wild-type alleles and found that the V133A mutant allele provides more than two-fold greater signal-to -background compared to the wild-type allele. With V133A, more extreme log₂FoldChange values are observed with greater separation between the positive and negative log₂FoldChange peaks. In other words, compared to wild-type, the V133A Dam-LMNB1 and Dam-only signals are more distinct. Kernel density estimate of log₂ Fold Change showed greater separation for cLAD and ciLAD signal with V133A. V133A has higher sensitivity than WT, with more differentially enriched regions at each log₂FoldChangethreshold for calling significant differential enrichment.

RNA sequencing was performed in bulk cells that were untreated or transfected with Dam-only, Dam-LMNB1, or ^{m6} A-Tracer, and only two differentially expressed genes were found. Differentially expressed genes compared to no treatment control are HIST2H4A and LIF for Dam, HIST2H4A for Dam-LMNB1, and no genes for ^{m6}A-Tracer. When comparing Dam to ^{m6} A-Tracer, the only differentially expressed gene is FKBP1A, which is expected given the mutated FKBP1A-derived destabilization domain tethered to Dam in our construct. When comparing Dam-LMNB1 to ^{m6} A-Tracer, the only differentially expressed gene is LMNB1, which is again expected given LMNB1 is expressed from the Dam-LMNB1 construct itself. This corroborates similar published findings that Dam expression and adenine methylation have little or no effect on gene expression in HEK293T cells (Park et al. 2018).

Three devices containing 25 imaged cells total were ran, with empty lanes left as negative controls, which did not yield sequenceable quantities of DNA. From these, 18 cells were selected for multiplexed sequencing, including 15 Dam-LMNB1 cells and 3 Dam-only cells, to achieve a desired level of coverage per cell. Selection was based on image quality and initial DNA quantification data from each sample (see methods herein). One anomalous Dam-LMNB1 cell was included that appeared to have high fluorescence in the nuclear interior, predicting that it might have higher background DamID coverage in non-LAD regions (cell #7). After sequencing, 3 Dam-LMNB1 cells containing a high fraction of sequencing reads mapping to the transfected plasmids were excluded; the 15 remaining cells had less than 5% of mapped reads mapping to plasmid DNA. For these 15 remaining cells, a median of roughly 600,000 raw reads per cell (range 300 k- 2.7 M) was obtained, covering a median of 110,000 unique DpnI fragments per cell (37 k - 370 k), in line with previous DamID results from single cells (Kind et al. 2015).

### B. µDamID Sequencing Data Recapitulate Existing LAD Maps

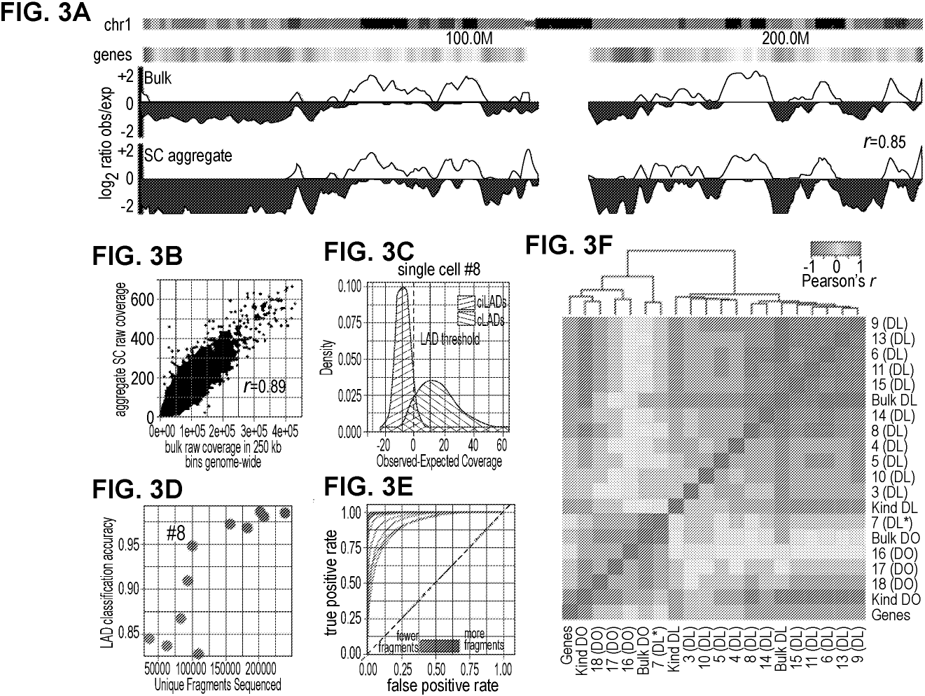

To assess whether single-cell µDamID sequencing data provide accurate measurements of lamina-associated domains, the single-cell results were compared to results obtained from bulk DamID in the same cell line. DamID results are reported as a difference or log ratio between the observed coverage from Dam-LMNB1 expressing cells and the expected coverage from background, estimated using coverage from Dam-only expressing cells (see methods described herein). This measure is reported within fixed 250 kb bins across the genome, which is half the median length of known LADs in the genome (Kind et al. 2015). By aggregating the data from 11 Dam-LMNB1 expressing cells passing filters and excluding the anomalous cell #7, excellent correspondence was found with the bulk data obtained from millions of cells (FIG. 3A), with a Pearson correlation of 0.85 across all bins in the genome. To ensure normalization is not inflating the correlation, aggregate single-cell raw read coverage was compared to bulk raw read coverage and observed a genome-wide correlation of 0.89 (FIG. 3B).

Pairwise correlations were next computed between the raw coverage for all single cells with each other, with the bulk data, with aggregated published single-cell DamID data (from Kind et al. 2015), and with the number of annotated genes in each 250 kb bin genome-wide. Unsupervised hierarchical clustering was performed on these datasets and produced a heatmap of their pairwise correlations (FIG. 3F). The 3 Dam-only single cells were found to cluster with each other, with the bulk Dam-only data, with the Kind et al. Dam-only data, and with the number of genes, as expected. The 11 Dam-LMNB1 cells cluster separately with each other, with the bulk Dam-LMNB1 data, and with the Kind et al. Dam-LMNB1 data. The anomalous cell #7 shows correlations with both the Dam-only and Dam-LMNB1 clusters, appearing intermediate between them (FIG. 3F). This illustrates that single-cell Dam-LMNB1 and Dam -only cells can be distinguished given their sequencing data alone, and they associate as expected with published data, with our bulk data, and with annotated gene density, further confirming that these sequencing data are measuring meaningful biological patterns in single cells. The anomalous cell #7 can also be distinguished by sequencing data alone, since its data correlate with both the Dam-only and Dam-LMNB1 cell data.

### C. µDamID Enables Accurate LAD Calling Within Single Cells

In order to define LADs across the genome within single cells, a simple classifier was trained on a set of stringent positive and negative controls: regions confidently known to be lamina-associated or not lamina associated based on bulk DamID data from our study and others (Lenain et al. 2017; see methods described herein). Positive controls consist of 250 kb bins across the genome that were previously annotated in other human cell lines and confirmed with bulk DamID in our own cell line to be consistently associated with the nuclear lamina (referred to as constitutive LADs, or cLADS). Negative controls were similarly determined using prior bulk data to be consistently not associated with the nuclear lamina (referred to as constitutive inter-LADs, or ciLADS). These stringent control sets constitute roughly 10% of the genome each.

For each single Dam-LMNB1 cell, the distribution of its normalized sequencing coverage in bins from the positive and negative control regions (FIG. 3C) was computed, with the expectation that ciLADs have little or no coverage and the cLADs have high coverage. Given these control distributions, a coverage threshold was chosen to maximally separate the known cLADs and ciLADs. Across the 11 Dam-LMNB1 cells, thresholds that distinguish the known cLADs and ciLADs with a median accuracy of 96% (range 83-99%) were determined, which correlates positively with the number of unique DpnI fragments sequenced per cell (FIG. 3D). Receiver operating characteristic (ROC) curves were plotted for each cell, showing the empirical tradeoff between false positive and false negative LAD calls at varying thresholds (FIG. 3E).

After choosing a threshold for each cell to maximize classification accuracy between the control sets, these thresholds were applied to make binary LAD classifications across the rest of the genome. At each bin in the genome, the number of Dam-LMNB1 cells were counted in which that bin was classified as an LAD (out of 11 total cells). As expected, bins belonging to the cLAD control sets are classified as LADs in almost all 11 of the cells while bins belonging to the ciLAD control sets are classified as LADs in almost none of the cells (FIGS. 4A, 4B). The intermediate bins (called as LADs in 4 to 7 cells), appearing to be lamina associated in only a subset of cells, are likely to contain regions that are variably associated with the lamina, differing from cell to cell, or possibly even dynamically moving between the lamina and the nuclear interior within the same cell over time (Kind et al. 2015). Single-cell data provide a unique opportunity to observe and measure this variability in chromatin organization between cells, enabling the identification of these variable LADs within a population of cells.

To classify bins confidently as variable LADs, the possibility that sampling error could explain the observed intermediate number of LAD-classified cells in these regions was considered, given the range of error rates within individual cells. Among bins called as LADs in 4-7 cells, the joint probability of observing that number of cells under two null models was computed: one consisting of true positives and false negatives, and one consisting of true negatives and false positives (see methods described herein). Only the subset of bins with low p-values (p<10⁻⁸) under both null models was selected, providing high confidence that these variable LAD regions are truly variable between cells (FIG. 4A). These stringently defined regions, which comprise 13% of the genome, were more gene rich and have higher gene expression than cLADs, given their dynamic positioning in cells. Indeed, these variable LADs show intermediate gene density and bulk gene expression levels compared to the control sets of cLADs and ciLADs (FIGS. 3C-3D), consistent with these regions being variably active within different cells.

### D. µDamID Enables Cell-Cell Comparisons Based on Imaging and Sequencing Data

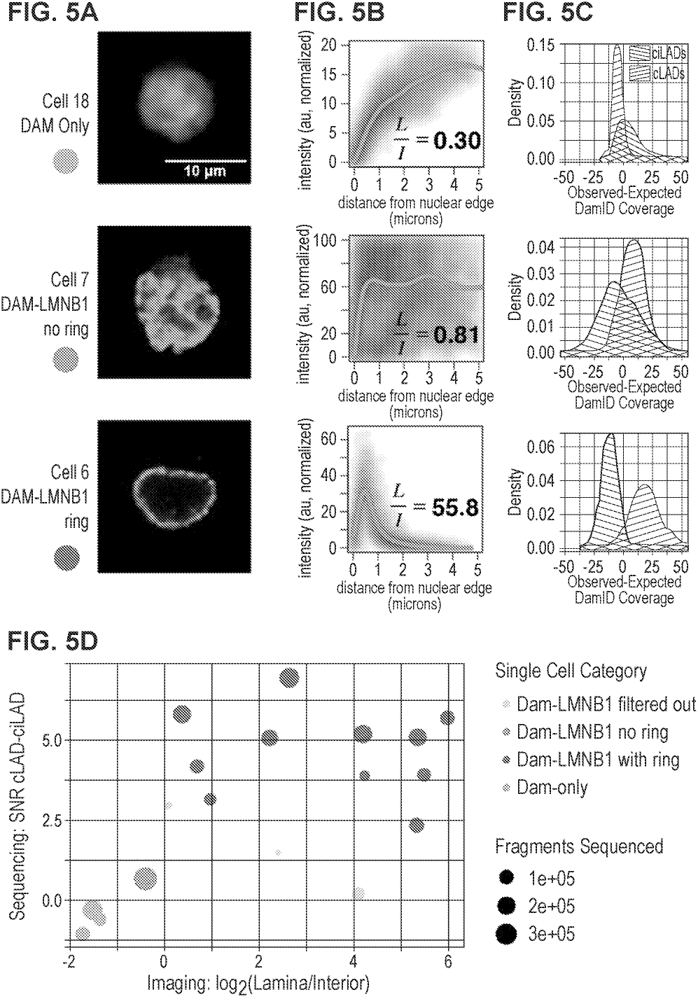

µDamID enables the joint analysis of the nuclear localization and sequence identity of protein-DNA interactions within each cell and between cells. Because the nuclear localization of LADs is well characterized, one could generate and test hypotheses about the sequencing data given the imaging data for each cell in this study. For example, cells expressing Dam-only show fluorescence throughout the center of the nucleus, and indeed their coverage profiles show little difference in coverage between known cLADs and ciLADs (FIGS. 5A-5C). Moreover, Dam-LMNB1 cells with visible rings and low fluorescence in the nuclear interior tend to show well -separated cLAD and ciLAD coverage distributions (FIGS. 5A-5C). One anomalous Dam-LMNB1 cell (cell #7) was selected for having bright fluorescence throughout the nucleus, and its sequencing data confirm that it appears to have increased coverage in ciLADs, appearing like an intermediate between the Dam-only and Dam-LMNB1 coverage signatures (FIGS. 5A-5C). Dam-LMNB1 is likely overexpressed in that cell, causing it to accumulate high background levels of methylation throughout the nucleus.

To quantify these observations across all cells, for each image we generated an averaged GFP intensity profile plot as a function of the distance from the edge of the nuclear lamina (FIG. 5B). Using these profiles, we computed the ratio of mean GFP intensity at the nuclear lamina compared to the nuclear interior, which is small for the Dam-only cells and cell #7, and large for the Dam-LMNB1 cells. Then, these imaging ratios were compared to a computed sequencing signal-to-noise ratio (SNR) for each cell, a measure of how well separated the cLAD and ciLAD coverage distributions are (see methods described herein and FIG. 5D). The Dam-only and Dam-LMNB1 cells can be readily separated on either axis, with cell #7 appearing intermediate on both axes. Overall, these data add additional confidence that the sequenced areas correspond to the fluorescing areas of the nucleus, providing two useful measures of chromatin organization within single cells.

### E. Imaging LADs in the µDamID Device Using m6A-Tracer-NES

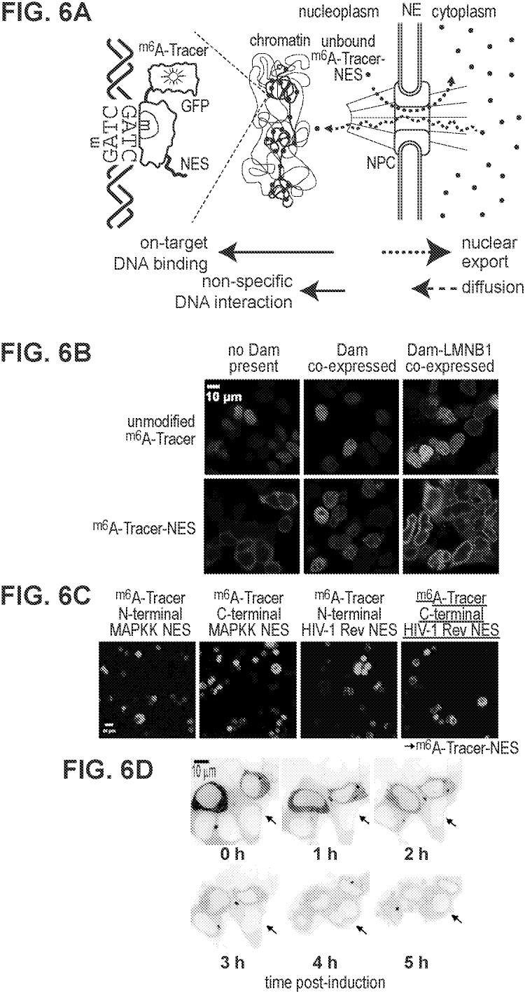

Fluorescence microscopy was used to quantify the spatial distribution of LADs in the µDamID device prior to DamID processing. In the first batch of 18 cells, m6A-Tracer was imaged to identify the localization of lamina-interacting DNA in the nucleus. Dam-LMNB1-expressing cells were selected that had laminar rings consistent with effective LAD methylation, as well as one anomalous Dam-LMNB1 cell with high signal in the nuclear interior (FIGS. 5A-5C). Fairly uniform fluorescence was observed across the nucleus in cells expressing untethered Dam. These imaging patterns were largely predictive of their respective sequencing coverage distributions (FIG. 5D). However, this investigation revealed an important aspect of the m6A-Tracer technology, which is that the m6A-Tracer protein localizes to the nucleus even in cells expressing no Dam (FIGS. 6A-6B). One consequence is that cells with Dam and cells without Dam are nearly indistinguishable (FIG. 6B top row), and cells with overexpressed m6A-Tracer show high background fluorescence levels in the nuclear interior even when co-expressing Dam-LMNB1 (FIG. 6B). The only way to prevent this background issue is to carefully tune the expression level of m6A-Tracer so that the copy number of m6A-Tracer proteins does not exceed the number of available methylated GATC sites. This tuning would have to occur separately for any new Dam fusion protein. In a heterogeneous expression system like the one used here, since m6A-Tracer and Dam are expressed from separate plasmids, only a small fraction of cells have the correct ratios of expression to produce sharp laminar rings with low background in the nuclear interior (FIG. 6B).

No cryptic nuclear localization sequences were detected in m6A-Tracer, nor are human cells likely to contain any significant background levels of m6A without Dam (O’Brown et al., (2019), BMC Genomics 20, 445. http://doi.org/10.1186/s12864-019-5754-6). Instead, its default nuclear localization may arise from a weak interaction between genomic DNA and the DNA binding domain of m6A-Tracer, combined with the ability of m6A-Tracer to diffuse freely through nuclear pores given its small size (FIG. 6A). Adding a Nuclear Export Signal (NES) to m6A-Tracer was hypothesized to overcome its weak affinity for DNA and keep any unbound copies of the protein sequestered in the cytoplasm. It was found that the HIV-1 Rev NES sequence fused to either terminus resulted in robust localization of m6A-Tracer to the cytoplasm in cells not expressing Dam (FIG. 6C), and for downstream experiments we proceeded to use the C-terminal fusion, which we call m6A-Tracer-NES.

While the NES appears to prevent nonspecific m6A-Tracer interactions with DNA, it does not overcome on-target binding to Dam-methylated DNA. When Dam was co-expressed, the localization of m6A-Tracer-NES shifted almost entirely from the cytoplasm to the nucleus (FIG. 6B). When Dam-LMNB1 was co-expressed, m6A-Tracer-NES shifted to the nuclear lamina, with excess copies remaining in the cytoplasm in a subset of cells with especially high expression (FIG. 6B,). This shift in localization began within 2-3 hours of Dam-LMNB1 induction and produced visible rings in the majority of transfected cells within 5 hours (FIG. 6D). Because m6A-Tracer-NES only binds methylated sites in the nucleus, it solves two major problems: 1) m6A-Tracer fluorescence in the nucleus is no longer ambiguous and can be interpreted as a signal of methylation, and 2) high contrast between the nuclear lamina and the nuclear interior can be achieved for a much wider range of m6A-Tracer expression levels.

As described herein, the use of an integrated microfluidic device for single-cell isolation, imaging, and sorting, followed by DamID was demonstrated. This system enables the acquisition of paired imaging and sequencing measurements of protein-DNA interactions within single cells, giving a readout of both the ‘geography’ and identity of these interactions in the nucleus. Specifically, the device was tested by mapping well-characterized interactions between DNA and proteins found at the nuclear lamina, providing a measure of genome regulation and 3D chromatin organization within the cell, and recapitulating similar maps in other cell types.

In some embodiments of the disclosure, this technology is applied to study other types of protein-DNA interactions in single cells, and is combined with other sequencing and/or imaging modalities to gather even richer information from each cell. For example, in one embodiment, the nuclear localization of specific proteins such as heterochromatin-associated proteins or nucleolus-associated proteins can be visualized by fluorescent tagging, then DamID is used to sequence and identify nearby genomic regions. Recent advances allow for simultaneous DamID and transcriptome sequencing in single cells (Rooijers et al., Nat Biotechnol. 2019 Jul;37(7):766-772), and the devices described herein could be adapted for similar multi-omic protocols as well. In other embodiments, the throughput of the platforms described herein are increased to hundreds of cells per device by scaling up the design and incorporating features like multiplexed valve control (Kim et al. 2017) and automated image processing and sorting. To scale this technology further, paired imaging and sequencing data could be obtained using spatially or optically registered DNA barcodes (Cole et al., Proceedings of the National Academy of Sciences of the United States of America, 114(33), 8728-8733, 2017; Nguyen et al., Advanced Optical Materials, 5(3), 1600548, 2017; Yuan et al., Genome Biology, 19(1), 227, 2018).

The above Examples demonstrate a real-time biosensor in a miniaturized, microfluidic device format that can continuously and simultaneously measure the concentration of molecules in a living subject with excellent sensitivity and less than a minute temporal resolution.

The various embodiments described above can be combined to provide further embodiments. All U.S. patents, U.S. patent application publications, U.S. patent application, foreign patents, foreign patent application and non-patent publications referred to in this specification and/or listed in the Application Data Sheet are incorporated herein by reference, in their entirety. Aspects of the embodiments can be modified if necessary to employ concepts of the various patents, applications, and publications to provide yet further embodiments.

These and other changes can be made to the embodiments in light of the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

What is claimed is:

1. A method of co-determining the cellular location and nucleotide sequence of a DNA that is contacted by a protein of interest in a single cell, said method comprising the steps of:
(a) incubating a collection of cells that express at least one protein of interest under conditions that allow the at least one protein of interest to contact a DNA sequence;
(b) isolating a single cell from the collection of cells and determining the cellular location of the DNA sequence within the single cell;
(c) amplifying and collecting the DNA comprising the DNA sequence; and
(d) determining the sequence of the DNA sequence;
wherein steps (b)-(c) are carried out in separate chambers within one lane of a microfluidic device.

2. The method of claim 1 wherein the incubating step (a) is carried out in a chamber within one lane of the microfluidic device.

3. The method of any of claims 1-2, wherein the DNA sequence comprises a DNA-binding site.

4. The method of any of claims 1-3, wherein the cells have been induced to express the protein of interest.

5. The method of claim 4, wherein the protein of interest is a recombinant protein and is expressed from an expression vector.

6. The method of any of claims 1-5 wherein the at least one protein of interest is selected from the group consisting of a nuclear lamina protein, a nucleolar protein, a transcription factor, a histone or histone variant, centromere protein A, a modification-specific internal antibody (mintbody), an intracellular scFV, a chromatin-modifying enzyme, an RNA polymerase, a DNA polymerase, a DNA helicase, a DNA repair protein, a Cas9 protein, a dCas9 protein, a zinc finger protein, a TALE protein, a CTCF protein, a cohesion protein, a synaptonemal complex protein, a telomere-binding protein, a centromere-binding protein, and an outer kinetochore protein.

7. The method of any of claims 1-6 wherein the at least one protein of interest has been engineered to modify one or more nucleotides at or near the DNA sequence.

8. The method of any of claims 1-7 wherein contacting the DNA sequence by the at least one protein of interest results in a modification to the DNA that is detectable by imaging.

9. The method of claim 8 wherein the modification is methylation.

10. The method of claim 6 wherein the methylation occurs at or near a sequence comprising GATC.

11. The method of claim 7 wherein the protein of interest is a fusion of the protein of interest and (i) DNA adenine methyltransferase (Dam) or a biologically active fragment thereof, or (ii) EcoGII methyltransferase or a biologically active fragment thereof.

12. The method of any one of claims 9-11 wherein the collection of cells that expresses the at least one protein of interest also expresses at least one imaging protein that binds to methylation sites.

13. The method of claim 12 wherein the imaging protein is a fusion of a protein that binds methylated DNA and a green fluorescent protein (GFP) or a biologically active fragment thereof.

14. The method of claims 13, wherein the imaging protein is m6a-Tracer or m6A-Tracer-NES.

15. The method of any of claims 1-14 wherein the cell is a bacterial cell, a eukaryotic cell or prokaryotic cell.

16. The method of claim 15 wherein the cell is a mammalian cell.

17. The method of claim 16 wherein the cell is a human cell.

18. The method of any of claims 1-17 wherein the cellular location of the DNA sequence contacted by the protein of interest is determined by a method selected from the group consisting of microscopy, confocal microscopy, confocal fluorescent microscopy, high resolution microscopy, scanning confocal microscopy, two-photon fluorescence microscopy, TIRF microscopy, lattice light-sheet microscopy, super-resolution microscopy, and Stochastic Optical Reconstruction Microscopy.

19. The method of any of claims 1-17 wherein the amplifying the DNA sequence of part (c) comprises the steps of (i) lysing the single cell, (ii) digesting DNA, (iii) ligating universal primers, and (iv) PCR amplification.

20. The method of claim 19 wherein each step (i) - (iv) is performed in a separate chamber.

21. The method of claim 19 wherein the lysing step comprises contacting the cell with a cell lysing agent selected from the group consisting of ionic and non-ionic detergents, Triton X-100, sodium dodecyl sulfate (SDS), NP-40, and ammonium chloride potassium.

22. The method of claim 19 wherein the digesting step comprises contacting the DNA from the lysed cell with a digesting agent selected from the group consisting of methyladenine-sensitive endoculease DpnI and methyladenine-sensitive endoculease DpnII.

23. The method of claim 22 wherein the agent is DpnI or a biologically active fragment thereof.

24. The method of any of claims 1-23 wherein the determining the sequence of the DNA sequence of step (d) allows the identification of an associated gene and/or locus within a genome.

25. The method of any of claims 1-24 wherein the microfluidic device comprises from 1-100 lanes.

26. The method of claim 23 wherein each lane of the microfluidic device can carry out steps (b)-(c) in parallel.

27. A method of co-determining the cellular location and nucleotide sequence of a DNA that is contacted by a protein of interest in a single cell, said method comprising the steps of:
(a) incubating a collection of cells that express a protein of interest under conditions that allow the protein of interest to contact a DNA sequence comprising a DNA-binding site;
(b) isolating a single cell from the collection of cells and determining the cellular location of the DNA comprising the DNA-binding site within the single cell;
(c) amplifying and collecting the DNA comprising the DNA-binding site; and
(d) determining the sequence of the DNA-binding site contacted by the protein of interest;
wherein steps (b)-(c) are carried out in separate chambers within one lane of a microfluidic device; wherein the protein of interest is a fusion of the protein of interest and Dam or a biologically active fragment thereof; wherein the cellular location of step (b) is determined by confocal fluorescent microscopy; wherein the amplifying the DNA sequence of part (c) comprises the steps of (i) lysing the single cell, (ii) digesting DNA, (iii) ligating universal primers, and (iv) PCR amplification; and wherein the cellular location of the DNA comprising the DNA-binding site of step (b) is coupled to the sequence of the DNA-binding site of step (d) to provide contemporaneous imaging and sequence measurement of a protein-DNA interaction.


SEQUENCE LISTING (8 sequences)

SEQ ID NO: 1 - protein, 9 aa, Artificial Sequence
  Synthetic Polypeptide
EQKISEEDL

SEQ ID NO: 2 - DNA, 42 bp, Artificial Sequence
  Synthetic Polynucleotide
ctaatacgactcactatagggcagcgtggtcgcggccgagga

SEQ ID NO: 3 - DNA, 10 bp, Artificial Sequence
  Synthetic Polynucleotide
tcctcggccg

SEQ ID NO: 4 - DNA, 23 bp, Artificial Sequence
  Synthetic Polynucleotide
  misc_feature (1)..(4): n is a, c, g, t or u
nnnngtggtcgcggccgaggatc

SEQ ID NO: 5 - DNA, 34 bp, Artificial Sequence
  Synthetic Polynucleotide
tacactctttccctacacgacgctcttccgatct

SEQ ID NO: 6 - DNA, 34 bp, Artificial Sequence
  Synthetic Polynucleotide
gtgactggagttcagacgtgtgctcttccgatct

SEQ ID NO: 7 - DNA, 15 bp, Artificial Sequence
  Synthetic Polynucleotide
ggtcgcggccgagga

SEQ ID NO: 8 - DNA, 15 bp, Artificial Sequence
  Synthetic Polynucleotide
tcctcggccgcgacc